(19) United States
(12) Patent Application Publication
(10) Pub. No.: US 2003/0216665 A1
(43) Pub. Date: Nov. 20, 2003

(54) Body composition measurement apparatus

(76) Inventors: Yoshihisa Masuo, Shiga-ken (JP); Kazuhiko Yoshida, Osaka-fu (JP)

Correspondence Address: ARMSTRONG,WESTERMAN & HATTORI, LLP, 1725 K STREET, NW, SUITE 1000, WASHINGTON, DC, 20006, US

(21) Appl. No.: 10/410,614

(22) Filed: Apr. 10, 2003

(30) Foreign Application Priority Data
Apr. 12, 2002 (JP) 2002-109931(P)

(51) Int. Cl. A61B 5/05

(52) U.S. Cl. 600/547

(57) ABSTRACT

The present invention proposes a body composition measurement apparatus which is easy to use and is still capable of accurately measuring body composition information, such as quantity and/or balance of body-fat, muscle mass of both thighs. In an embodiment of the invention, the apparatus has a body 10 having tapered parts 11L and 11R designed to support the legs at a bending angle of about 160 degrees when the legs are laid on the tapered parts. Measuring electrodes 14L and 14R at the top of the tapered parts contact the backs of the knees. Grip bars 12L and 12R projecting from both sides of the body 10 has measuring electrodes 15L and 15R, which contacts both palms of the subject when the subject holds the grip bars 12L and 12R with both hands. Current-carrying electrodes 13L and 13R provided on the slopes of the tapered parts contact both calves (or lower thighs). Supplying electric current through the current-carrying electrodes between both lower thighs, the apparatus measures the voltage induced over each of both thighs, and calculates the impedance of each thigh. Then, using estimation formulae created beforehand based on the regression analysis of data collected by an MRI method, the apparatus estimates body composition, such as the muscle masses of both thighs and its right-and-left balance, from the measurement value of the impedance and body identification information including the height, weight, etc.

The present invention relates to a body composition measurement apparatus for measuring bioelectrical impedance (simply referred to as “impedance” hereinafter) of the body of a subject to estimate and present various kinds of information about the body composition and/or health condition of the subject. Particularly, the present invention relates to a body composition measurement apparatus which conveniently allows the subject to be in a sitting or supine position. The apparatus uses not only the measurement value of the impedance but also the height, weight, age, sexuality and other information (which will be generally referred to as “body identification information” hereinafter), to estimate the body-fat mass, muscles, muscle force, bone density, bone mass, lean body mass, body-fat ratio, basal metabolic rate and other information (which will be generally referred to as “body composition information” hereinafter).

### BACKGROUND OF THE INVENTION

Conventionally, the commonest method in health management concerning obesity or other body conditions is to measure the body weight. Nowadays, obesity is not regarded simply as a body type, but people are looking at indices for measuring obesity. One such index is the body-fat mass, which indicates the mass of subcutaneous fat and/or visceral fat; another is the body-fat ratio, which indicates the ratio of body-fat to body weight.

Many parties have been studying methods of measuring the impedance of the body and estimating body-fat ratio and/or other values based on the impedance. One such method is a four-electrode method, which uses a pair of current-carrying electrodes attached to the back of the right hand and the instep of the right foot of the subject, respectively, and a pair of measuring electrodes attached to some parts between the current-carrying electrodes, such as the right wrist and the right ankle. With a radio-frequency current being supplied through the body between the current-carrying electrodes, the potential difference between the measuring electrodes is measured. Then, the impedance of the body is calculated from the potential difference and the current value, and the body-fat ratio and/or other information is estimated based on the impedance.

Recently, more convenient apparatuses for measuring body-fat ratio (so-called “body-fat meters”) have been developed, and are commercially available. An example of such apparatuses is disclosed in the Japanese Unexamined Patent Publication No. H7-51242. This apparatus includes a pair of grips to be held by both hands, each grip provided with a current-carrying electrode and a measuring electrode. The electrodes are located so that, when the subject holds the grips with both hands, the current-carrying electrode contacts the hand at a part close to the fingers and the measuring electrode contacts the hand at a part close to the wrist. Then, based on the bioelectrical measured impedance thereby, the apparatus estimates various kinds of information about the living body, such as lean body mass, body-fat ratio, total body water or basal metabolic rate. Another example is disclosed in the Japanese Examined Patent Publication No. H5-49050, which has the above electrodes located to contact the soles of the feet when the subject steps on it, so that the weight and the body-fat can be simultaneously measured.

The above-described apparatuses measure the impedance with the current path extending between one hand and one foot, between both hands or between both feet. In the case of measuring the potential difference with the current path between one hand and one foot, the current path includes the chest or abdomen (i.e. trunk) whose cross-sectional area is a good deal larger than that of a leg or an arm. In this case, the contribution of the leg or the arm to the impedance is relatively great while that of the subcutaneous fat of the abdomen or the intra-abdominal fat (visceral fat) is relatively small. This means that the measurement result hardly reflects the increase or decrease in the subcutaneous fat of the abdomen or the intra-abdominal fat, so that the result lacks reliability. In the case of measuring the potential difference with the current path between both hands or both feet, on the other hand, the trunk is not included in the current path, so that the error in the estimation of the body-fat ratio or other indices for the entire body is likely to be large.

Another problem relates to an estimation formula used to estimate body-fat ratio or other indices from measurement values of the impedance. Conventionally, such a formula is created according to a calibration curve prepared based on the result of an underwater weighing method. By this method, however, it is impossible to decrease the estimation error because the method has some faults, such as lack of consideration of the difference in the contribution of the muscle, bone or other lean body components to the impedance.

Still another problem relates to a prerequisite for the application of the above measurement method. The body composition is calculated from the impedance on the assumption that the human body can be modeled as a structure composed of three tissues: bone, muscle and fat, which have different electrical characteristics. In this model, it is assumed that the three tissues are connected in parallel, the component ratios of the tissues are fixed, and the whole component tissues and each tissue have fixed electrical characteristics (i.e. volume resistivity). Various statistical researches suggest that this assumption is highly reliable for normal adult people. However, for children, minors, aged people, athletes or other groups of people having special body composition, it is impractical to obtain reliable results because the personal differences in the component ratios and the electrical characteristics are so great that the values often deviate from the above condition.

In respect not of the prevention of obesity, but in respect of the determination of the progress of strengthening of the body or the progress of aging, it is very important to measure the muscle mass or muscle force of the body. For example, an athlete or similar person who intends to improve the ability of the body will not only regard the muscle mass as an index for measuring the effect of training or other activities but also use the index to set an objective for training. This observation applies also to the case of a person in the process of rehabilitation for the strengthening and recovery of a part of the body that has weakened after a long hospital stay due to an injury or illness. Furthermore, the expected increase of aged people will make it necessary to measure the muscle mass, muscle force and right-and-left balance of such properties of the muscle of each aged person in the nursing care. The information obtained by the measurement will make it possible to evaluate the ability of the person to live an independent life, and to provide an improved living environment and diet plan (meals, exercises) that are designed to supplement any inconvenience or shortage in daily life so that the person can exhibit a high performance in daily activity.

What is important to satisfy the above requirements is that ordinary people can easily measure the various kinds of body composition information, such as muscle mass, at home as well as in hospitals or sports facilities (e.g. fitness clubs), not to speak of the necessity of accurate measurement of body composition information. That is, it is desirable that even such people that have no special skill for measurement can carry out the measurement by themselves without taking any unnatural position. It is also desirable that measurement apparatuses are available at low prices. Additionally, the apparatus may be preferably portable, occupying only a small storage space.

ADL (Activity of Daily Life, or Living) evaluation is one of the known methods of evaluating the ability of aged persons, or persons subjected to medical treatments because of illness or injury, to live a physically independent life. Examples of ADL evaluation are the Barthel Index and the Functional Independence Measure (FIM). To compare the results of such evaluations performed at different nursing facilities, or to maintain the absoluteness of evaluation, it is desirable to establish a more objective method of ADL evaluation.

According to the ADL evaluation, one important checkpoint is whether the person is able to walk independently. This ability is known to be mostly dependent on the quadriceps, the muscle located at the front of the thigh. The quadriceps easily weakens with aging. The weakening of the quadriceps makes it hard for the person to lift the knees; the person may stumble over a very low step, or feel it difficult to go up and down stairs. If there is a great difference in muscle force between the right and left quadriceps, it will impose a burden to the pelvis or joints and may cause an imbalance between the right and left sides of the body. Once abraded, the joint cannot be reproduced. This means that an abrasion of a joint at one side of the body due to an unbalanced burden may determine the life of the person. Therefore, it is profitable to accurately measure the mass and force of the quadriceps to provide the subject with an ADL index and appropriate advisory information based on the ADL index.

The present invention has been achieved in view of the above problems, the principal object of which is to propose a body composition measurement apparatus which is easy to use and is still capable of accurately measuring various kinds of body composition information, such as quantity and/or balance of body-fat, muscle mass, muscle force, bone mass, bone density and other indices. Particularly, the present invention proposes a body composition measurement apparatus which can be easily used to obtain the muscle mass of lower limbs or other body composition information that is useful for the ADL evaluation.

### SUMMARY OF THE INVENTION

Conventional measurement apparatuses, such as weighing machines, often require the subject to take a standing position. However, the standing position makes some muscles swelled or strained to maintain that position, which greatly affects the measurement. Taking this into account, the body composition measurement apparatus according to the present invention is designed to allow the subject to take a sitting or supine position during the measurement. Such positions allow the subject to be relaxed so that the muscles of the lower limbs are only slightly burdened. This makes it possible to accurately measure the muscle mass, bone density or other indices of the lower limbs.

Thus, according to a first aspect of the present invention, the body composition measurement apparatus includes:

a) a positioning supporter for supporting a joint located at an end of a predetermined target part of the body of a subject at a preset angle;

b) an electrode holder for fixedly or movably holding plural electrodes so that the electrodes contact predetermined parts of the body of the subject;

c) an impedance-measuring device for measuring the impedance of the target part with the electrodes; and

d) an estimator for estimating various kinds of information about body composition and/or health condition of the target part or entire body of the subject, based on the measured impedance.

The “various kinds of information relating to the body composition and/or health condition” includes: body-fat mass (or ratio), lean body mass (or ratio), total body water (or body water ratio), bone mass (or ratio), muscle force, muscle density, degree of obesity, basal metabolic rate, energy metabolism, and ADL index for measuring the activity of daily life. The above quantities and ratios may be calculated for the entire body or each part of the body. The quantities and ratios may be also used to check the balance between the right and left parts, between the upper and lower parts and/or between the distal and proximal parts.

In the apparatus according to the first aspect of the invention, the target part is such a part of the body that can be approximately modeled as a column having a certain length and composed of tissues with their cross-sectional area ratios almost fixed. Examples of the target part are as follows: the part of an arm between the wrist and the shoulder (or acromion); the part of a leg between the ankle and the groin (or trochanterion); and the trunk (or idiosoma). It is more preferable to divide the arm into two target parts: forearm and upper arm. Similarly, the leg may be preferably divided at the knee into two target parts: crus (or lower thigh) and thigh. The part of the upper limb between the wrist and the roots of the fingers at the back of the hand may be chosen as a target part. Similarly, the part of the lower limb between the ankle and the roots of the fingers at the instep of the foot may be chosen as a target part. Any of the aforementioned parts may be further divided into smaller parts. For example, a part of the right or left forearm around the wrist and/or a part of the crus around the ankle may be chosen as a target part.

To estimate the muscle mass or other quantities of the target part, the sizes of the target part, such as the length or cross-sectional area, are used as variables. The lengths and cross-sectional areas of the flexors and extensors of the lower limb depend on the bending angle of the knee. Similarly, the lengths and cross-sectional areas of the flexors and extensors of the upper limb depend on the bending angle of the elbow. Therefore, it is difficult to achieve high reproducibility of measurement without maintaining the joint of the knee or elbow at a fixed angle. It is preferable to maintain the knee or elbow slightly bent rather than straightened. This positioning makes it possible to assuredly locate the front and back parts across the joint, to determine the contact positions of the electrodes without difficulty, and to prevent the displacement of the electrodes. The correct positioning of the electrodes improves the accuracy of the measurement of impedance.

In the apparatus according to the first aspect of the invention, the positioning supporter fixedly holds or supports the joint of the knee, elbow or other part at a preset angle. This angle is determined so that the subject can easily bend the part to that angle without feeling any burden; there shall not be any need to forcefully bend that part of the body. The electrode holder fixedly or movably holds the electrodes so that the electrodes contact predetermined points that are suitably defined for measuring the impedance of a target part, at an end of which a joint is supported as described above. Using the electrodes, the impedance-measuring device measures the impedance of the target part. Based on the impedance, the estimator estimates the body composition information corresponding to the target part and/or the body composition of the entire body.

Thus, by using the apparatus according to the first aspect of the invention, the cross-sectional areas of the body component tissues, such as muscular tissue, fatty tissue and osseous tissue, are maintained almost unchanged throughout the measurement. This makes it possible to accurately estimate various kinds of information relating to the body composition and/or health management corresponding to the target part, or such information of the entire body of the subject. This information makes it possible to give the subject appropriate advice concerning health management, health improvement, rehabilitation or training.

Particularly, the apparatus may be constructed to measure a target part defined by a joint, such as knee or elbow, at an end, or defined by a pair of right and left joints at both ends. This construction provides a small-sized, easy-to-use apparatus, making the measurement easy to perform. The construction also reduces the production costs of the apparatus.

Among the component tissues of the body, the flexors and extensors of the upper or lower limb greatly change their length and cross-sectional area depending on the bending angle of the elbow or knee. Therefore, the measurement accuracy can be greatly improved by fixing the joint at a natural angle during the measurement.

Thus, according to a second aspect of the present invention, the body composition measurement apparatus includes:

a) a positioning supporter for supporting a joint located at an end of a predetermined target part of the body of a subject at a preset angle;

b) an electrode holder for fixedly or movably holding plural electrodes so that the electrodes contact predetermined parts of the body of the subject;

c) an impedance-measuring device for measuring the impedance of the target part with the electrodes; and

d) an estimator for estimating muscle mass of the target part or entire body of the subject, based on the measured impedance.

The above body composition measurement apparatus can accurately measure the muscle mass without difficulty. This apparatus provides appropriate information that serves as indices for training or other activities, and also provides high motivation, to those who intend to improve their body ability or who intend to recover some physical capability through rehabilitation therapy. This apparatus can be used to measure the muscle force or right-and-left balance of the quadriceps, which easily weakens with aging. This measurement provides an index for determining whether an aged person, or a person subjected to a medical treatment because of illness or injury, can live a physically independent life. In addition, based on such determination, it is possible to provide a guide for an improved living environment and diet plan (meals, exercises) that are designed to supplement any inconvenience or shortage in daily life.

In general, on the assumption that the bone volume does not change with aging, the water content in the bone increases as the calcium and other highly insulating minerals decrease with aging. This change lowers the electrical characteristic, or impedance, of the bone. Accordingly, the bone density, particularly the decrease in the bone density with aging, can be accurately measured based on the impedance.

Thus, according to a third aspect of the present invention, the body composition measurement apparatus includes:

a) a positioning supporter for supporting a joint located at an end of a predetermined target part of the body of a subject at a preset angle;

b) an electrode holder for fixedly or movably holding plural electrodes so that the electrodes contact predetermined parts of the body of the subject;

c) an impedance-measuring device for measuring the impedance of the target part with the electrodes; and

d) an estimator for estimating bone density of the target part or entire body of the subject, based on the measured impedance.

In the measurement using the above apparatus, information about osseous tissue can be accurately measured by selecting, as the target part, the ankle, wrist or other part of the subject where the bone tissue occupies a relatively large cross-sectional area. The apparatus according to the third aspect of the invention can be used to accurately measure the bone density of aged people, or patients suffering from a particular disease, who need to pay attention to the deterioration of the osseous tissue. For those kinds of people, it is often difficult to keep a standing position or keep their arms raised forward. In the measurement using the apparatus according to the third aspect of the invention, the subject experiences little physical burden, because there is no need to take an unnatural position or to use any great muscle force to maintain the joint fixed at a preset angle.

In a mode of the apparatus according to any one of the first to third aspects of the invention, the impedance-measuring device is constructed based on an approximate model where the impedance of a part of the body is represented by impedance elements connected in parallel, including impedance elements corresponding to fatty tissue, muscular tissue and osseous tissue, respectively, and where the entire body of the human being is separated into body parts, in which the component ratios of the tissues are fixed and the whole component tissues and each tissue have fixed electrical characteristics, and the impedance-measuring device further includes:

c1) plural current-carrying electrodes and plural measuring electrodes, to be attached to the body of the subject to measure the impedance of the target part composed of one body part or two or more body parts connected in series;

c2) a current-supplying device for supplying an alternating current of a preset frequency via the current-carrying electrodes through at least the target part;

c3) a voltage-measuring device for measuring the voltage induced by the alternating current over the target part; and

c4) a calculator for calculating the impedance corresponding to the target part from the voltage measured and the current value of the alternating current.

The estimator d) may be constructed to estimate the muscle mass, the bone density or various kinds of information relating to the body composition or health condition of the target part or entire body of the subject, using:

a first estimation formula created based on the result of the measurement of the impedance for the entire body and/or each body part of each of plural pretest subjects, and based on the body composition information of the entire body and/or each body part of each of the pretest subjects obtained by observing the inside of the entire body and/or each body part of each of the pretest subjects; or

a second estimation formula created by adding specific body information of the pretest subjects to the first estimation formula.

Means for “observing the inside of the entire body and/or each body part of each of the pretest subjects” is preferably constructed to perform a non-destructive observation. Examples of such means are the nuclear magnetic resonance imaging apparatus (MRI) and the computed tomography (CT) scanner; these apparatuses can acquire cross-sectional images of the inside of a body from the outside. For example, the MRI can take cross-sectional images of the abdominal cavity, arms, legs or other body parts at preset intervals. From these cross-sectional images, the masses and occupation ratios of the living body tissues (fat, muscle, bone, etc.) in a given part of the body can be obtained by identifying each living body tissue in every cross-sectional image, calculating the mass and occupation ratio of each tissue, and integrating the results of the analysis of all the cross-sections included in the given part. The accuracy of the estimation formula can be improved by performing the above measurement based on the observation for a number of pretest subjects (or monitors) of different height, weight, age and sexuality, measuring the impedance corresponding to each body part of each pretest subject, and creating an estimation formula based on the measurement results.

The apparatus according to any one of the first to third aspects of the invention may further include a body identification information acquirer for acquiring body identification information of the subject, and the estimator estimates the muscle mass, bone density or various kinds of information relating to the body composition or health condition of the target part or entire body of the subject, based on the measured impedance and the body identification information.

The body identification information typically includes the following information about the body type of the subject: height, weight, and partial sizes of a body part, such as the length and/or circumference of a leg. The information may also include the age and/or sexuality of the subject. Further, the information may include other kinds of information that affect the body and its health, such as the personal history of illness and injury. These kinds of information are greatly correlated with the body composition. Therefore, the accuracy of estimation can be improved by referencing such information.

The body identification information may be manually entered into the apparatus by the subject or by an operator. In addition, the body identification information acquirer may include a partial size estimator for estimating a predetermined size or sizes of the target part based on the height of the subject externally given as one item of the body identification information, and adding the estimated size or sizes to the body identification information. This estimation may also take into account the weight, age, sexuality or other information. Also, the body identification information acquirer may include a size measurer for measuring the actual size of the target part of the subject. The size measurer provides a size or sizes of the target part more accurate than the size or sizes estimated as described above. As a result, the accuracy of estimation of the body composition information is improved.

Usually, in the impedance measurement or the internal body measurement using the MRI, the subject takes a supine position with the joints at the knees, elbows and other parts straightened; that is, with the joints maintained at angles of almost 180 degrees. This positioning makes the estimation formula more accurate because the sizes (length and cross-sectional area) of the muscle of, for example, the thigh differs little between the impedance measurement and the internal body measurement. When an estimation formula obtained as described above is used to estimate the body composition of a subject, the best condition for minimizing the estimation error is that the subject takes the same position as pretest subjects during the measurement. This is because such positioning makes the bending state of the flexors and extensors within the body of the subject the same as that of the pretest subject. Thus, to estimate the body composition of the subject with high accuracy using an estimation formula, the best positioning is to maintain the joints of the knees and elbows at almost 180 degrees of angle. It should be noted that the basic idea for the above positioning is to “straighten” the knees and elbows in a natural way; it is not always necessary to maintain the joint accurately at 180 degrees of angle.

For aged people or those who have poor flexibility in their body, it may be difficult to take a sitting position with their legs straightened so that the joints are maintained at 180 degrees of angle. The subjects often feel more comfortable when they slightly bend the knees to relax the muscles at the backs of the knees. In addition, as will be described later, the accuracy of measuring the impedance is affected by the contact positions of the electrodes attached to the body of the subject. Therefore, it will be difficult to ensure the reproducibility of the measurement if the accuracy of positioning the electrodes is low. When the knee is straightened, it is difficult to determine the contact positions of the electrodes. When, on the other hand, the knee is slightly bent, it is easier to determine the contact positions of the electrodes at the back of the leg across the knee. However, the measurement accuracy will be too low if the lengths and cross-sectional areas of the muscles in the thigh and crus come off the aforementioned condition as a result of bending the knee.

In general, when the knee is bent from 180 degrees by about 20 degrees, the length and cross-sectional area of the muscle located before and behind the knee change less than when the knee is greatly bent (to angles of 120-110 degrees or less, for example). Thus, the influence of the change in the angle can be minimized by bending the joint to about 160 degrees. This angle not only satisfies the above condition but also brings the subject into a relatively comfortable position. In addition, the electrodes can be positioned with adequately high accuracy. However, the subject usually feels more comfortable when the knee is bent a little more than that angle. Accordingly, when the comfortableness is more important than the measurement accuracy, it will be preferable to bend the knee to about 140 degrees.

The above observation suggests that the angle of the joint should be 180 degrees from the point of view of the accuracy of estimation, while the angle should be within a range from about 140 to 180 degrees to ensure the easiness (or comfortableness) of the measurement for the subject and the accuracy of positioning the electrodes. Accordingly, the positioning supporter may preferably maintain the joint of the subject at a preset angle within a range from about 140 to 180 degrees. It should be noted that the range does not include the 180 degrees, where the joint is fully straightened. The joint should be bent intentionally and slightly.

To maintain the above position for measurement, the positioning supporter may be constructed to support the lower limbs at least at the backs of the knees when the subject is in a sitting position with the lower limbs stretched forward. For example, the positioning supporter may include a trapezoidal or triangular flat body having slopes for resting the lower limbs. Another example of the positioning supporter has an approximately horizontal bar located at a predetermined level for supporting the backs of the knees.

For aged or medically ill individuals, it may be difficult to take a sitting position on the floor with the legs straightened forward. Therefore, in some cases, it may be more desirable to allow the subject to take a more comfortable position, even if it decreases the measurement accuracy. For such a case, the positioning supporter may be constructed to maintain the joint of the subject at an angle of about 90 degrees. To maintain such a position, the positioning supporter may be constructed like a chair having a seat for the subject to sit down, where the level of the seat is determined so that the knees of the subject are bent to the almost right angle while the soles of the feet are placed on the floor or other approximately horizontal plane equivalent to the floor. This construction allows the subject to take a position more comfortable than sitting on the floor during the measurement.

In a mode of any one of the first to third aspects of the invention, the measuring electrodes include a pair of electrodes that contact the proximity to the knees of the subject and at least one electrode that contacts the trunk or upper limb of the subject, and the current-carrying electrodes include a pair of electrodes that contact the body at two points located farther than the knees from the trunk of the subject. There, at least one measuring electrode that contacts the trunk or upper limb may preferably contact a palm of the subject. With this construction, the natural action of the subject to grasp an object produces a sure contact of the measuring electrode to the palm. The current-carrying electrodes may be designed to contact the calves of the subject.

The apparatus constructed as described above supplies a weak alternating current via the current-carrying electrodes through at least both thighs (i.e. the target parts). This current induced voltages in both thighs, respectively. No potential difference is produced along the voltage measurement path within such parts of the body where the above electric current does not flow. This means that such parts can be regarded as mere lead wires as far as the measurement of the voltage is concerned. For example, the section between the palm and the lower limb (or the groin at which the thighs are connected, strictly speaking) may be regarded as a mere lead wire whose impedance can be ignored. Therefore, the potential difference between the right palm and the right knee can be regarded as equivalent to the potential difference due to the impedance of the right thigh. From the voltage value thus measured and the current value, the impedance of the right thigh can be calculated.

The impedance calculated as described above corresponds to a unitary body part whose impedance can be approximately represented by a model where the impedance elements corresponding to the fatty tissue, muscular tissue and osseous tissue, respectively, are connected in parallel, and where the component ratios of the tissues are fixed and the whole component tissues and each tissue have fixed electrical characteristics. The body parts thus sectioned are rather strictly consistent with the model that serves as a basis for calculating the body composition, i.e. the model that is constructed using the results of measurements using an MRI. Therefore, the estimation can be performed with high accuracy for each body part represented by the above model.

In the above mode of the invention, the estimator may estimate at least the muscle mass of the thigh of the subject or other body composition information correlated with the muscle mass of the thigh. The estimator may estimate at least the balance of the muscle mass of both thighs of the subject or other body composition information correlated with the above balance. The estimator may use the length of the thigh estimated based on the body identification information.

In the measurement relating to the muscle of the thigh, the measurement cannot be performed with high reproducibility unless the knee is maintained at a predetermined angle, as explained above. Furthermore, the knee should be slightly bent rather than stretched, because such positioning can fix the knee at a desired position and hence facilitate the positioning of the measuring electrodes.

For example, when the knee is bent to an angle determined within the range from about 140 to 180 degrees, the kneecap comes to the top of the bending part. In this state, the aforementioned measuring electrodes that contact the proximity to the knees may be preferably arranged to contact the backs of the knees, whereby the measuring electrodes are accurately positioned.

The measuring electrodes that contact the proximity to the knees may be located at the top the positioning supporter having a trapezoidal or triangular flat body with slopes for resting the lower limbs, and the current-carrying electrodes that contact the calves may be located on the slope. Alternatively, the aforementioned measuring electrodes may be located on the upper side of the horizontal bar for supporting the knees, and the aforementioned current-carrying electrodes may be arranged to contact the backs of the calves at a level lower than the bar.

In another mode of the invention, the measuring electrodes that contact the proximity to the knees are arranged to contact the surfaces of the knees. The kneecap bulges out a little even when the leg is almost straightened, to say nothing of when the leg is bent. This bulging of the kneecaps can be used to assuredly determine the contact positions of the measuring electrodes with respect to the surface of the knees.

The measuring electrodes that contact the proximity to the knees may be arranged to contact the insides of the knees when the apparatus is held between the knees. This arrangement uses the closing force of the legs of the subject so that the measuring electrodes tightly contact the insides of the knees.

The electrode that contacts a palm may be a grip-like electrode to be gripped by the subject, which serves as the electrode holder with a handle for pulling the grip-like electrode out from the body of the apparatus.

In another mode of the invention, the measuring electrode that contacts a palm is a grip-like electrode, which serves as the electrode holder when connected to the body by a cable. Alternatively, the measuring electrode that contacts a palm may be provided on each of both sides of the body of the apparatus so that the measuring electrodes contact both palms when the subject touches both sides of the body of the apparatus with both hands.

The electrode holder may include a position adjuster for adjusting the contact positions of the measuring electrodes. This construction makes it possible to adjust the measuring electrodes to predetermined contact positions, irrespective of the body type of the subject, so that the measurement can be performed with high accuracy. The position adjuster may include a size-measuring device for measuring a size or sizes of the target part according to the contact positions adjusted. This construction eliminates the necessity to manually enter the size or sizes of the target part as one item of the body identification information, so that the measurement work is simplified. Further, this construction improves the accuracy of estimation of the body composition information because it prevents erroneous entries of the size of the target part while obtaining accurate values by actual measurement.

In a mode of the apparatus according to any one of the first to third aspects of the invention, the apparatus includes a square-shaped body housing containing an electrical circuit, and the measuring electrodes are arranged at preset intervals on both sides, bottom side or other side adjacent to the aforementioned sides of the body housing so that the measurement can be performed with the knees set apart from each other by a preset distance. This construction assuredly prevents the legs from contacting, which would negatively affect the measurement. The body housing may be further provided with a display on its front side or upper side. The display can be used, for example, to show information about operation and input, and various kinds of information about the measurement result by using characters, graphs, etc.

In another mode of the invention, the electrode holder is constructed to allow the subject to grip and move the electrode holder to any position on the body, and the impedance-measuring device measures the impedance elements corresponding to different contact parts of the measuring electrode held by the electrode holder. This construction is advantageous for cost reduction because at least one electrode is commonly used in the measurements of several parts of the body.

In another mode of the invention, the impedance-measuring device includes a first unit for principally measuring the upper limbs of the subject and a second unit for principally measuring the lower limbs of the subject, and a cable connects the first and second units for transferring signals between the two units. It is also possible to use a wireless communication system to transfer signals between the first and second units. The wireless communication system may use radio waves, light, ultrasonic waves, etc. The use of the cable is advantageous for cost reduction, and the use of the wireless communication system is advantageous for easy handling of the apparatus.

In another mode of the invention, the positioning supporter includes a stimulating apparatus for giving a stimulus to at least a part of the body of the subject. The term “stimulus” hereby means anything that desirably affects the living body tissue of a part or entire body of the subject. For example, the stimulus may strengthen the living body tissue, or promote the blood circulation or metabolism, to contribute to the improvement in health condition.

For example, the stimulating apparatus may be a massager for massaging at least a part of the body of the subject. In this case, the positioning supporter may be constructed as a chair having the massager. This construction makes it easy to check the effects of the massage on the promotion of blood circulation or the elimination of swelling by performing the above-described measurements before and after the massaging. Thus, the user can efficiently improve the health condition or recover the health.

### BRIEF DESCRIPTION OF THE DRAWINGS

FIG. 1 shows an approximate model of the impedance configuration of the human body according to the measurement method applied to the body composition measurement apparatus according to the present invention.

FIG. 2 shows a simplified version of the approximate model of FIG. 1, which is applied to practical measurements.

FIG. 3A conceptually shows the cross-sectional images of a body part acquired with an MRI, and FIG. 3B shows an area distribution of the tissues along the longitudinal direction of the body part.

FIG. 4A shows a columnar composition model used in the measurement method according to the present invention, and FIG. 4B shows the equivalent circuit.

FIG. 5 is a perspective view of the body composition measurement apparatus as the first embodiment of the present invention.

FIG. 6 is a perspective view of the body composition measurement apparatus of the first embodiment being used in a measurement.

FIG. 7 is a side view of the body composition measurement apparatus of the first embodiment being used in a measurement.

FIG. 8 shows the configuration of the electrical system of the body composition measurement apparatus of the first embodiment.

FIG. 9 is a flowchart showing the steps of the measurement using the body composition measurement apparatus of the first embodiment.

FIG. 10 shows an approximate model of the impedance configuration of the human body, which is applied to measurements using the body composition measurement apparatus of the first embodiment.

FIG. 11 is a perspective view of a variation of the body composition measurement apparatus of the first embodiment.

FIG. 12 is a side view of the measurement apparatus of FIG. 11 being used in a measurement.

FIG. 13 is a perspective view of another variation of the body composition measurement apparatus of the first embodiment.

FIG. 14 is a perspective view of the measurement apparatus of FIG. 13 being used in a measurement.

FIG. 15 is a perspective view of another variation of the body composition measurement apparatus of the first embodiment.

FIG. 16 is a perspective view the measurement apparatus of FIG. 15 being used in a measurement.

FIG. 17 is a perspective view of another variation of the body composition measurement apparatus of the first embodiment.

FIG. 18 is a perspective view of another variation of the body composition measurement apparatus of the first embodiment.

FIG. 19 is a top view of the measurement apparatus of FIG. 18 being used in a measurement.

FIGS. 20A-20B show a body composition measurement apparatus as the second embodiment of the present invention, where FIG. 20A is a top view and FIG. 20B is a bottom view.

FIG. 21 is a side view of the body composition measurement apparatus of the second embodiment being used in a measurement.

FIGS. 22A-22B show a variation of the body composition measurement apparatus of the second embodiment, where FIG. 22A is a front view and FIG. 22B is a bottom view.

FIG. 23 shows a side view of the measurement apparatus shown in FIGS. 22A-22B being used in a measurement.

FIG. 24 is a perspective view of another variation of the body composition measurement apparatus of the second embodiment.

FIG. 25 is a top view of the measurement apparatus of FIG. 24 being used in the measurement.

FIGS. 26A-26B show a body composition measurement apparatus as the third embodiment of the present invention, where FIG. 26A is a side view and FIG. 26B is a top view.

FIG. 27 is a side view of the measurement apparatus of FIGS. 26A-26B being used in a measurement.

FIG. 28 is an enlarged perspective view of a foot under measurement performed using the measurement apparatus of FIGS. 26A-26B.

FIG. 29 is a perspective view of the first measurement unit of a body composition measurement apparatus as the fourth embodiment of the present invention.

FIGS. 30A-30B show the second measurement unit of the body composition measurement apparatus of the fourth embodiment, where FIG. 30A is a side view and FIG. 30B is a top view.

FIG. 31 is a side view of the body composition measurement apparatus of the fourth embodiment being used in a measurement.

FIG. 32 shows the configuration of the electrical system of the first measurement unit.

FIG. 33 shows the configuration of the electrical system of the second measurement unit.

FIG. 34 is a perspective view of a body composition measurement apparatus as the fifth embodiment of the present invention.

FIG. 35 is a perspective view of a part of the body composition measurement apparatus of the fifth embodiment.

FIG. 36 is a perspective view of the body composition measurement apparatus of the fifth embodiment being used in a measurement.

FIG. 37 is a perspective view of a variation of the body composition measurement apparatus of the fifth embodiment.

FIG. 38 is an enlarged view of a part of the measurement apparatus of FIG. 37.

FIG. 39 is a partial side view of the measurement apparatus of FIG. 37 being used in a measurement.

FIG. 40 is a perspective view of another variation of the body composition measurement apparatus of the fifth embodiment.

FIG. 41 is a perspective view of a part of the measurement apparatus of FIG. 40.

FIG. 42 is an enlarged view of a part of a body composition measurement apparatus as the sixth embodiment of the present invention.

FIG. 43 is a perspective view of the body composition measurement apparatus of the sixth embodiment being used in a measurement.

FIG. 44 is a side view showing the state of the measurement using a body composition measurement apparatus as the seventh embodiment of the present invention.

FIG. 45 is a perspective view of another variation of the body composition measurement apparatus of the first embodiment.

FIG. 46 is a side view of the measurement apparatus of FIG. 45 being used in a measurement.

FIG. 47 is a side view of another variation of the body composition measurement apparatus of the first embodiment being used in a measurement.

### DETAILED DESCRIPTION OF A PREFERRED EMBODIMENT

The construction and operation of the body composition measurement apparatus according to the present invention will be described in detail. To begin with, the measurement method applied to the body composition measurement apparatus according to the present invention is explained.

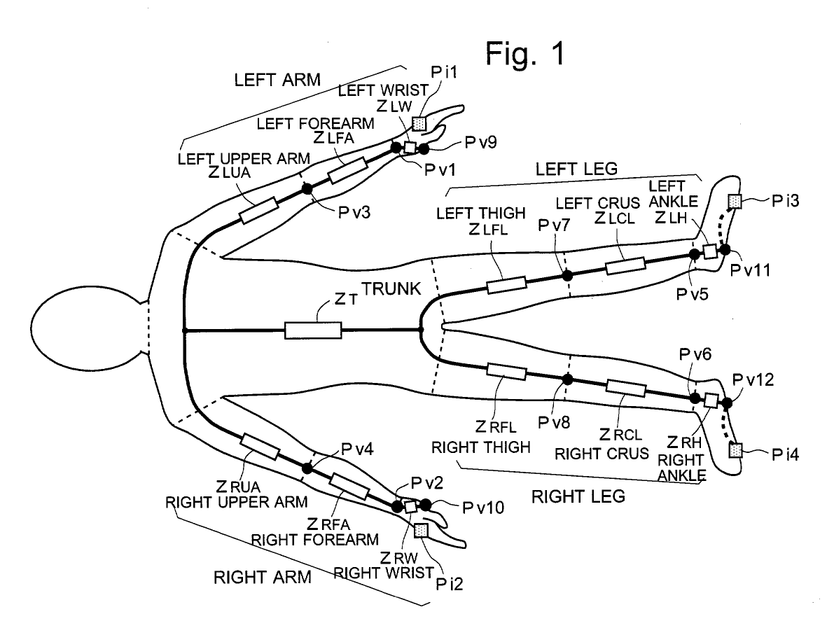

FIG. 1 shows an approximate model showing the impedance configuration of the human body according to the present measurement method. According to this method, the human body is divided into plural segments, and the impedance is calculated for each segment or for two or more segments connected in series. To improve the accuracy of estimation of the body composition information based on the impedance, the segment is defined corresponding to every part of the body where the composition of the tissue is relatively uniform. The segment thus defined can be approximately represented by a columnar model, which will be explained later.

For example, except for the head, and the fingertips of the hands and the feet, the body is divided into thirteen segments: left wrist, left forearm, left upper arm, right wrist, right forearm, right upper arm, left thigh (or left regio femoralis), left calf, left ankle (or left heel), right thigh (or right regio femoralis), right calf, right ankle (or left heel), and trunk. Each of the thirteen segments has certain impedance. Therefore, a body can be represented as a model composed of plural impedance elements connected as shown in FIG. 1. In FIG. 1, Z_{LW}, Z_{LFA}, Z_{LUA}, Z_{RW}, Z_{RFA}, Z_{RUA}, Z_{LFL}, Z_{LCL}, Z_{LH}, Z_{RFL}, Z_{RCL}, Z_{RH} and Z_{T} denote the impedance values of the above-listed thirteen segments, respectively.

To measure the impedance of the thirteen segments, four current-supplying points (Pi1 to Pi4) and twelve voltage-measuring points (Pv1 to Pv12) are defined on the limbs of the subject. The current-supplying points Pi1 to Pi4 are located at or close to the roots of the middle fingers on the backs of both hands and at or close to the roots of the middle fingers on the insteps of both feet. The voltage-measuring points Pv1 to Pv12 are located at the palms of both hands, at the wrists and elbows of both arms, at the bottoms of both heels and at the ankles and knees of both legs.

When electric current is supplied between the two points selected from the four current-supplying points Pi1 to Pi4, the potential difference between a predetermined pair of the voltage-measuring points can be regarded as a potential voltage induced between the ends of an impedance element, or between the ends of plural impedance elements connected in series. The current hardly flows through such parts of the body that are not on the current path. Therefore, the segments corresponding to such parts of the body can be regarded as mere lead wires whose impedance can be ignored.

Suppose the current is supplied between the current-supplying points Pi3 and Pi4 located at the feet. In this case, the potential difference between the voltage-measuring points Pv5 and Pv6 located at both ankles is equal to the voltage corresponding to the impedance composed of Z_{LCL}, Z_{LFL}, Z_{RFL} and Z_{RCL} connected in series; i.e. the impedance of both legs. The potential difference between the voltage-measuring points Pv7 and Pv8 located at both knees is equal to the voltage correspond to the impedance including Z_{LFL} and Z_{RFL}. The potential difference between the voltage-measuring point Pv9 located at the left palm and the voltage-measuring point Pv7 located at the left knee is equal to the voltage corresponding to the impedance Z_{LFL} of the left thigh, because the left arm and the trunk can be regarded as mere lead wires.

It is clear that the measurement can be similarly performed for different current-supplying points, voltage-measuring points and body parts. Thus, by the above measurement method, the impedance can be measured accurately and independently for every segment.

The basic concept of the present measurement method is to independently obtain the impedance of each of the thirteen segments. When, however, the measurement should be performed in a simplified manner, it will be difficult to attach the four current-supplying points and the twelve voltage-measuring points on the body of the subject. One possible approach to this problem is to group plural neighboring segments connected in series as one segment. In this case, the measurement can be performed with smaller number of current-supplying points and voltage-measuring points. When, on the other hand, the measurement aims to obtain body composition information about a particular part of the body, e.g. muscle mass of the thigh, then the measurement of the impedance of only that part of the body will suffice. Thus, it is not always necessary to provide all the sixteen points mentioned above; in the minimal case, two current-supplying points and two voltage-measuring points will suffice to measure the impedance of a desired part of the body.

Methods of estimating body composition information based on the impedance obtained as described above are explained below. The estimation methods described hereby are characterized in that they use estimation formulae, developed based on body composition information acquired with an MRI, to estimate body composition information based on the measured impedance and the body identification information.

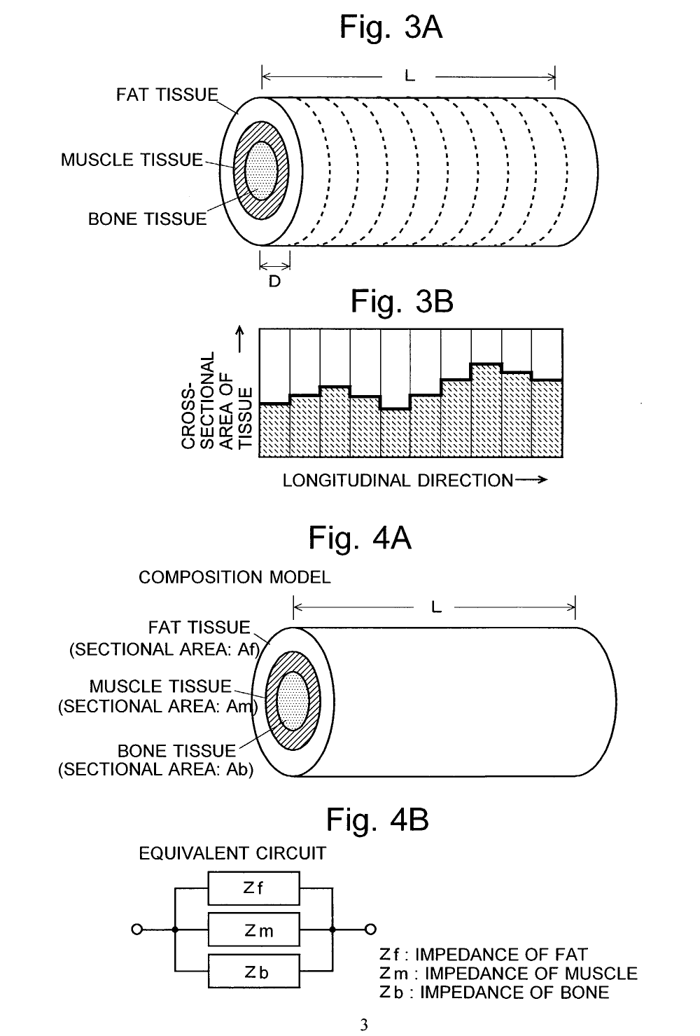

With an MRI, it is possible to acquire cross-sectional images of any part of the human body. The cross-sectional images provide information about the masses and/or ratios of the tissues, such as muscle, fat and bone, within the cross section. FIG. 3A schematically shows the cross-sectional images acquired at intervals D (10 mm, for example) along the longitudinal direction of the body part concerned. From these images, the masses (or areas) of the tissues, such as muscle, fat and bone, can be calculated. From this calculation, a diagram showing the area distribution of the tissues over the longitudinal direction of the body part can be created, as shown in FIG. 3B. Then, the areas are integrated along the longitudinal direction to determine the masses of the tissues within the body part concerned. According to the present invention, the body is divided into thirteen segments, so that it is easy to perform the MRI measurement for each unitary segment. Furthermore, since each segment is defined so that it approximates to a columnar body, the mass of the tissue can be obtained with high accuracy.

Important examples of the method of estimating body composition information are as follows.

[1] Estimation of Composition of the Entire Body

The “composition” hereby includes: body-fat ratio (%Fat), lean body mass (LBM), fat mass (FM), etc.

[1-1] Example of Estimation of Total Body-Fat Ratio

Based on the study of Lukaski et. al., the following formula has been conventionally used to estimate lean body mass by bioelectrical impedance methods:

LBM [kg]=a₀+b₀×(H²/Z₁)+c₀×W+d₀×Ag,

where a₀, b₀, c₀ and d₀ are constants (multiple regression coefficients) whose values differ according to the sexuality, and H, W, Ag and Z₁ are height, weight, age and impedance between wrist and ankle, respectively.

Using the lean body mass (LBM) and the weight (W), the body-fat ratio (%Fat) is given by:

%Fat=[(W−LBM)]/W]×100,

and the fat mass (FM) is given by:

FM=W−LBM.

Instead of using the above formula, the lean body mass (LBM) may be calculated by another method, which will be described later.

[1-2] Example of Estimation of Total Lean Body Mass

Regarding each of the thirteen segments of the body is regarded as a columnar model, the body composition of the segment is estimated by either of the following two methods.

[1-2-1] First method: Consider four limbs and trunk as five segment units, and create multiple regression formula using the five segment units as independent variables.

For the sake of simplification, the entire body is hereby divided into five segments: i.e. four limbs and trunk. Denoting the total lean body mass as LBM, lean body mass of both arms as LBMₕ, lean body mass of both legs as LBM_{L}, and lean body mass of the trunk as LBMₜᵣ, the following formulae are obtained:

LBMₕ∝Hₕ²/Zₕ

where Hₕ is the length of one or both arms and Zₕ is the impedance of one or both arms,

LBM_{L}∝H_{L}²/Z_{L}

where H_{L} is the length of one or both legs and Z_{L} is the impedance of one or both legs,

LBMₜᵣ∝Hₜᵣ²/Zₜᵣ

where Hₜᵣ is the length of the trunk and Zₜᵣ is the impedance of the trunk.

From these formulae, the following formula is obtained:

LBM=a₀+b₀×Hₕ²/Zₕ+c₀×H_{L}²/Z_{L}+d₀×Hₜᵣ²/Zₜᵣ+e₀×W+f₀×Ag (1),

where weight (W) and age (Ag) serve as supplementary variables for improving the correlation. The term of “Ag” corrects the difference in properties of the tissues depending on the age, and the term of “W” compensates for the influences on the characteristics, such as bone density, due to the stress of the weight on the osseous tissue. Naturally, the values of the coefficients a₀, b₀, c₀, d₀, e₀ and f₀ differ depending on the sexuality.

In general, Hₕ, H_{L} and Hₜᵣ are highly correlated with the height (H) of the subject. Accordingly, formula (1) can be rewritten as follows by substituting H for Hₕ, H_{L} and Hₜᵣ:

LBM=a₀′+b₀′×H²/Zₕ+c₀′×H²/Zₕ+d₀′×H²/zₜᵣ+e₀′×W+f₀′×Ag (2).

Here, Zₕ may be either the impedance of both arms or the impedance of one arm. In the case of one arm, it is assumed that both arms have the same impedance. The same argument also holds true for Z_{L}. Alternatively, the average of the impedance values of both arms, or both legs, may be used as Zₕ, or Z_{L}.

When the four limbs are regarded as independent, then formula (1) will be rewritten as follows:

$$\begin{matrix}LBM = {a}_{0}^{″} + {b}_{0}^{″} \times {H}_{h R}^{2} / {Z}_{h R} + {c}_{0}^{″} \times {H}_{h L}^{2} / {Z}_{h L} + {d}_{0}^{″} \times {H}_{L R}^{2} / {Z}_{L R} + \\ {e}_{0}^{″} \times {H}_{L L}^{2} / {Z}_{LL} + {f}_{0}^{″} \times {H}_{t r}^{2} / {Z}_{t r} + {g}_{0}^{″} \times W + {h}_{0}^{″} \times Ag ,\end{matrix} \qquad ( 3 )$$

where

H_{hR} is the length of the right arm, and Z_{hR} is the impedance of the right arm,

H_{hL} is the length of the left arm, and Z_{hL} is the impedance of the left arm,

H_{LR} is the length of the right leg, and Z_{LR} is the impedance of the right leg, and

H_{LL} is the length of the left leg, and Z_{LL} is the impedance of the left leg.

Furthermore, when the measurement can be performed for each of the thirteen segments, formula (1) will be rewritten as follows:

$$\begin{matrix}LBM = {a}_{0} + {b}_{0} \times {H}_{UAR}^{2} / {Z}_{UAR} + {c}_{0} \times {H}_{FAR}^{2} / {Z}_{FAR} + {d}_{0} \times {H}_{UAL}^{2} / {Z}_{UAL} + \\ {e}_{0} \times {H}_{FA L}^{2} / {Z}_{FAL} + {f}_{0} \times {H}_{FLR}^{2} / {Z}_{FLR} + {g}_{0 \times } {H}_{CLR}^{2} / {Z}_{CLR} + {h}_{0} \times \\ {H}_{FLL}^{2} / {Z}_{FLL} + {i}_{0} \times {H}_{CL L}^{2} / {Z}_{CLL} + {j}_{0} \times {H}_{t r}^{2} / {Z}_{t r} + {k}_{0} \times W + {l}_{0} \times Ag .\end{matrix} \qquad ( 4 )$$

It should be noted that any of the above formula (1), (2), (3) and (4) does not necessarily include all the variables as shown above; the formula may be preferably composed of only independent variables that are essentially effective. The above formulae should be regarded as examples where the maximal number of variables are used.

[1-2-2] Second method: Estimate body composition for each segment, and include the estimated values into the formula for estimating the total body composition.

Denoting the lean body mass of both arms as LBMₕ, the lean body mass of both legs as LBM_{L}, and the lean body mass of the trunk as LBMₜᵣ, the following formulae are obtained:

$$\begin{matrix}LBM = {a}_{0} + {b}_{0} \times {LBM}_{h} + {c}_{0} \times {LBM}_{L} + {d}_{0} \times {LBM}_{tr} , \\ {LBM}_{h} = {a}_{1} + {b}_{1} \times {H}_{h}^{2} / {Z}_{h} + {c}_{1} \times W + {d}_{1} \times Ag , \\ {LBM}_{L} = {a}_{2} + {b}_{2} \times {H}_{L}^{2} / {Z}_{L} + {c}_{2} \times W + {d}_{2} \times Ag , \\ {LBM}_{tr} = {a}_{3} + {b}_{3} \times {H}_{tr}^{2} / {Z}_{tr} + {c}_{3} \times W + {d}_{3} \times Ag .\end{matrix} \qquad ( 5 )$$

Formula (5) corresponds to formula (1). It is also possible to create formulae corresponding to formulae (3) and (4).

[1-3] Estimation of Total Muscle Mass and Total Bone Mass

Conventional anatomic data or similar data generally suggests that total muscle mass (TMM) is about 50% of lean body mass (LBM), and total bone mass (TBM) is about 16% of weight (W) or about 18% of lean body mass (LBM). Using these values, it is easy to roughly estimate the total muscle mass (TMM) and the total bone mass (TBM) from the lean body mass (LBM) and weight (W) obtained as described above.

Total muscle mass (TMM) and total bone mass (TBM) have significant correlation with lean body mass (LBM). Therefore, it is also possible to create multiple regression formula using variables similar to those used in the estimation formulae of LBM.

TMM=a₀+b₀×H²/Z₁+c₀×W+d₀×Ag

TBM=a₁+b₁×H²/Z₁+c₁×W+d₁×Ag

The above formulae are the simplest ones. As explained above, it is possible to create more complex formulae for more accurate estimation.

[2] Estimation of Body Composition of Each Segment Unit

[2-1] Estimation of Lean Body Mass

The columnar composition model as shown in FIG. 4A is applied to each segment.

That is, each segment includes fatty tissue having cross-sectional area A_{f}, muscular tissue having cross-sectional area Aₘ and osseous tissue having cross-sectional area A_{b}. These 10 tissues have the same length L. Denoting the volume resistivity of the fatty tissue, muscular tissue and osseous tissue as ,,_{f},,ₘ and ,,_{b}, respectively, the impedance Z_{f}, Zₘ and Z_{b} are given as follows:

Z_{f}=,,_{f}×(L/A_{f}),

Zₘ=,,ₘ×(L/Aₘ),

Z_{b}=,,_{b}×(L/A_{b}).

The impedance Z₀ of the segment concerned can be approximately represented by a model composed of Z_{f}, Zₘ and Z_{b} connected in parallel, as shown in FIG. 4B. Accordingly, the impedance Z₀ is given as follows:

1/Z₀=(1/Z_{f})+(1/Zₘ)+(1/Z_{b}) (11).

Now, the volume and density of non-fat layer (the layer other than the fat layer or panniculus) are denoted by V_{LBM} and D_{LBM}. The density D_{LBM} is known from conventional studies. Then, the lean body mass (LBM) is given as follows:

LBM=V_{LBM}×D_{LBM}.

where

V_{LBM}=A_{LBM}×L=(Aₘ+A_{b})×L=,,ₘ×(L²/Zₘ)+,,_{b}×(L²/Z_{b}) (12).

From formulae (11) and (12), Zₘ can be cancelled as follows:

V_{LBM}=,,ₘ×L²×[1/Z₀−1/Z_{f}]+(,,_{b}−,,ₘ)×(L²/Z_{b}) (13).

where the volume resistivity values of the tissues satisfy the condition: ,,ₘ<,,_{b}<<,,_{f}.

It is assumed hereby that there is no influence of distal parts such as wrists or ankles (Condition A). If this is true, then

A_{b}<<Aₘ.

Therefore,

Z_{f}(=,,_{f}×L/A_{f})>Z_{b}(=,,_{b}×L/A_{b})>>Zₘ(=,,ₘ×L/Aₘ)>Z₀.

If this relation is applied to formula (13), then

V_{LBM}=,,ₘ×(L²/Z₀)+(,,_{b}−,,ₘ)×(L²/Z_{b}) (14).

Here,

,,ₘ×(L²/Z₀)>>(,,_{b}−,,ₘ)×(L²/Z_{b}),

so that

V_{LBM}=,,ₘ×(L²/Z₀),

and

LBM=D_{LBM}×,,ₘ×(L²/Z₀).

Therefore, LBM can be expressed by a predetermined function f(x) as follows:

LBM=f(L²/Z₀).

Now, the influences of distal parts, such as wrists or ankles, are taken into account (Condition B). In this case,

A_{b}<Aₘ.

Therefore

,,ₘ×(L²/Z₀)>(,,_{b}−,,ₘ)×(L²/Z_{b})=,,V_{b}.

In general, the greater the weight is, the greater the volume V_{b} of the osseous tissue becomes to support the body. Accordingly, the following relation can be assumed: V_{b}∝,, V_{b}∝f(W). Therefore, from formula (14),

V_{LBM}=,,ₘ×(L²/Z₀)+(,,_{b}−,,ₘ)×(L²/Z_{b})=,,ₘ×(L²/Z₀)+,, V_{b}≈,,ₘ×(L²/Z₀)+(f(W),

so that

LBM=f(L²/Z₀, W).

Taking into account the change of tissues with aging and the difference depending on the sexuality, the estimation formula for multiple regression analysis can be created as follows:

LBM=a″+b″×(L²/Z₀)+c″×W+d″×Ag (15),

where a″, b″, c″ and d″ are constants (multiple regression coefficients), which take different values for different sexuality. The values of a″, b″, c″ and d″ for each sexuality can be determined beforehand by applying the lean body mass (LBM) obtained by an MRI method to the estimation formula for multiple regression analysis.

[2-2] Estimation of Muscle Mass

Estimation of muscle mass can be performed basically in the same manner as the above-described estimation of lean body mass. Denoting the volume and density of the muscle layer as V_{MM} and D_{MM}, respectively, the muscle mass is given as follows:

MM=V_{MM}×D_{MM}.

Using the impedance Zₘ of the muscle layer,

V_{MM}=,,ₘ×(L²/Zₘ).

Under the condition A,

MM≈LBM=a+b×(L²/Z₀)+c×Ag (16).

Under the condition B, on the other hand,

LBM=MM+BM=a+b×(L²/Z₀)+c×W+d×Ag (17).

In formula (17), the term of L²/Z₀ contains information about bone (BM) in addition to the muscle mass (MM); it is impossible to separate them. Among nine segments, exclusive of both wrists and both ankles, the upper arms and thighs satisfy the condition A, and the forearms and calves satisfy the condition B.

It is known that, for each person, the muscle mass of the upper arm is highly correlated with that of the forearm, and the muscle mass of the thigh is highly correlated with that of the calf. Therefore, it is possible to obtain information about muscle mass of upper arm (MM_{U}) and information about muscle mass of forearm (MM_{F}). That is, based on the regression analysis of MM_{UA} and MM_{FA} obtained by an MRI method, the following estimation formula can be created:

MM_{FA}=aₘ+bₘ×MM_{UA} (18).

Similarly, muscle mass of lower calf (MM_{CL}) can be estimated from the information about muscle mass of thigh (MM_{FL}) obtained by an MRI method:

MM_{CL}=a′ₘ+b′ₘ×MM_{FL} (19).

Muscle masses of a proximal segment, such as upper arm or thigh, can be obtained by formula (16) because such a segment satisfies the condition A. Then, using the muscle mass of the upper arm and that of the thigh in formula (18) and (19), respectively, the muscle mass of the forearm and that of the calf can be estimated.

[2-3] Estimation of Bone Mass

Paying attention to the forearm and the calf, both satisfying the condition B, the bone masses BM_{FA} and BM_{CL} can be calculated by subtracting MM_{FA} and MM_{CL}, calculated by formula (18) and (19), from the lean body masses LBM_{FA} and LBM_{CL}, calculated by formula (15), respectively:

BM_{FA}=LBM_{FA}−MM_{FA} (20),

BM_{CL}=LBM_{CL}−MM_{CL} (21).

Based on the bone masses obtained by formulae (20) and (21), the bone masses of other segments, each satisfying the condition A, and the total bone mass are estimated. Similar to the case of muscle mass, the bone mass of the upper arm is highly correlated with that of the forearm, and the bone mass of the thigh is highly correlated with that of the calf. Accordingly, based on the regression analysis of BM_{FA} and BM_{CL} obtained by an MRI method, the following estimation formulae can be created:

BM_{UA}=a_{b}+b_{b}×BM_{FA} (22),

BM_{FL}=a′_{b}+b′_{b}×BM_{CL} (23).

Similarly, it is possible to create estimation formulae based on the total bone mass and the regression analysis of arms and legs by an MRI method.

The above estimation method assumed that the lean body mass, muscle mass, muscle force, bone mass, etc., are estimated for each segment. In some cases, the result becomes more accurate when the estimation formulae are created so that the lean body mass, muscle mass, muscle force, bone mass, etc., are estimated for each unit length within the segment. This method is particularly effective in the case where the subject is an athlete or other type of person who has such a special body type that the segment lengths or other sizes of the upper arm and forearm, or those of the thigh and calf, are greatly unbalanced between the right and left sides of the body.

An example of estimation of muscle mass and bone mass per unit length is described below. The volume (V), cross-sectional area (A) and length (L) of a columnar model satisfy the following relation:

V=A×L.

Therefore,

V/L=A=,,×(L/Z).

Formulae (16)-(23) can be rewritten into per-unit-length forms as follows:

$$\begin{matrix}MM / L \approx LBM / L = a + b \times ( L / {Z}_{0} ) + c \times Ag , & {( 16 )}^{′} \\ LBM / L = ( MM + BM ) / L = a + b \times ( L / {Z}_{0} ) + c \times W + d \times Ag , & {( 17 )}^{′} \\ {MM}_{FA} / {L}_{FA} = {a}_{m} + {b}_{m} \times {MM}_{UA} / {L}_{UA} , & {( 18 )}^{′} \\ {MM}_{CL} / {L}_{CL} = {a}_{m}^{′} + {b}_{m}^{′} \times {MM}_{FL} / {L}_{FL} , & {( 19 )}^{′} \\ {BM}_{FA} / {L}_{FA} = {LBM}_{FA} / {L}_{FA} - {MM}_{FA} / {L}_{FA} , & {( 20 )}^{′} \\ {BM}_{CL} / {L}_{CL} = {LBM}_{CL} / {L}_{CL} - {MM}_{CL} / {L}_{CL} , & {( 21 )}^{′} \\ {BM}_{UA} / {L}_{UA} = {a}_{b} + {b}_{b} \times {BM}_{FA} / {L}_{UA} , & {( 22 )}^{′} \\ {BM}_{FL} / {L}_{FL} = {a}_{b}^{′} + {b}_{b}^{′} \times {BM}_{CL} / {L}_{CL} . & {( 23 )}^{′}\end{matrix}$$

Therefore,

$$\begin{matrix}{MM}_{UA} = ( {MM}_{UA} / {L}_{UA} ) \times {L}_{UA} , \\ {MM}_{FA} = ( {MM}_{FA} / {L}_{FA} ) \times {L}_{FA} , \\ {MM}_{FL} = ( {MM}_{FL} / {L}_{FL} ) \times {L}_{FL} , \\ {MM}_{CL} = ( {MM}_{CL} / {L}_{CL} ) \times {L}_{CL} , \\ {LBM}_{FA} = ( {LBM}_{FA} / {L}_{FA} ) \times {L}_{FA} , \\ {LBM}_{CL} = ( {LBM}_{CL} / {L}_{CL} ) \times {L}_{CL} , \\ {BM}_{UA} = ( {BM}_{UA} / {L}_{UA} ) \times {L}_{UA} , \\ {BM}_{FA} = ( {BM}_{FA} / {L}_{FA} ) \times {L}_{FA} , \\ {BM}_{FL} = ( {BM}_{FL} / {L}_{FL} ) \times {L}_{FL} , \\ {BM}_{CL} = ( {BM}_{CL} / {L}_{CL} ) \times {L}_{CL} ,\end{matrix}$$

Expressions using the function f(x) are as follows:

$$\begin{matrix}{MM}_{UA} = f ( {L}_{UA}^{2} / {Z}_{UA} ) , or f ( {L}_{UA}^{2} / {Z}_{UA} , W , Ag ) , \\ {MM}_{FL} = f ( {L}_{FL}^{2} / {Z}_{FL} ) , or f ( {L}_{FL}^{2} / {Z}_{FL} , W , Ag ) , \\ {MM}_{FA} = f ( {L}_{FA}^{2} / {Z}_{FA} , {L}_{UA}^{2} / {Z}_{UA} , W , Ag ) , or f ( {L}_{FA}^{2} / {Z}_{FA} , {L}_{UA}^{2} / {Z}_{UA} , W , Ag ) \times {L}_{FA} , \\ {MM}_{CL} = f ( {L}_{CL}^{2} / {Z}_{CL} , {L}_{FL}^{2} / {Z}_{FL} , W , Ag ) , or f ( {L}_{CL}^{2} / {Z}_{CL} , {L}_{FL}^{2} / {Z}_{FL} , W , Ag ) \times {L}_{CL} .\end{matrix}$$

[3] Estimation of Basal Metabolic Rate

A general method of estimating basal metabolic rate (BM) is as follows:

BM [kCal]/day≈RM/1.2∝VO₂r [mL/min]∝LBM [kg]∝TMM [kg],

where RM is resting metabolic rate, VO₂r is resting oxygen intake, LBM is lean body mass and TMM is total muscle mass. For example, when LBM=59.9 kg, then

VO₂r=(LBM+7.36)/0.2929=229.635 [mL/min].

If respiratory quotient (RQ) is 0.82 (constant), one liter of oxygen gas produces 4.825 kCal of heat. Accordingly, the daily oxygen consumption is:

229.635[mL/min]×60[min]×24[hr]=330.674[L],

and the basal metabolic rate (BM) is:

BM=4.825 [kCal]×330.674=1595.5[kCal].

The following discussion focuses on muscles as constituent tissues of the lean body mass (LBM). The present measurement method can accurately estimate the muscle mass of each segment. Therefore, it is expected that basal metabolic rate (BM) and resting metabolic rate (RM) can be estimated more accurately by using total muscle mass (TMM) rather than lean body mass (LBM). Taking this into account, multiple regression formulae may be created as follows:

BM (or RM)=f(TMM),

or

BM (or RM)=f(,,[MM of each segment]).

Furthermore, it is expected that muscles of different parts of the body have different degrees of contribution to the basal metabolism. For example, the muscles of legs will contribute to the basal metabolism more than the muscles of arms. This suggests that the muscle mass of legs (thighs and calves) is more correlated with the basal metabolic rate (BM) and the resting metabolic rate (RM) than the total muscle mass (TMM). Taking this into account, multiple regression formulae may be created as follows:

BM (or RM)=f(MM_{FL}, MM_{CL}).

Conventionally, fatty tissue was not taken into consideration because it contributes little to the basal metabolism. It is true that fat tissue is less active than muscular tissue, but it can still contribute to metabolism to some extent. Therefore, in order to improve the estimation accuracy, it is desirable to create estimation formulae that take into account fatty tissues. Accordingly, using fat mass (FM), multiple regression formulae may be created as follows:

BM (or RM)=f(TMM, FM).

It is said that the basal metabolic rate, particularly that of women, is not always highly correlated with the lean body mass; it is rather correlated with the weight. This suggests that the metabolism of fat tissue is not ignorable. The present measurement method can accurately estimate fat mass (FM). By using this fat mass, it is possible to effectively improve the accuracy of estimation of basal metabolic rate.

[4] Estimation of ADL Index

In this embodiment, muscle mass of quadriceps, maximum muscle force of quadriceps and weight-supporting index are used as the ADL index, but other indices may be used. Muscle mass of quadriceps is highly correlated with muscle mass of a leg or thigh, which includes quadriceps as its component. Therefore, muscle mass of quadriceps can be easily estimated from that of a leg or thigh calculated as described above. The maximum muscle force of quadriceps can be easily estimated from the muscle mass of the quadriceps. Furthermore, weight-supporting index can be estimated from the maximum muscle mass of quadriceps and the body weight.

As described above, the present measurement method makes it possible to estimate information that reflects the body composition or health condition, such as mass of tissue or basal metabolic rate, from measurement values of the impedance, based on the regression analysis of masses of tissues obtained by an MRI method.

Examples of the construction and operation of the body composition measurement apparatus according to the present invention using the above-described measurement method are described below. The measurement apparatuses of the following embodiments are constructed so that the muscle mass and muscle force of lower limbs, mainly thighs, can be accurately measured without difficulty.

As explained above, the following conditions are important to perform measurements by MRI methods with high accuracy:

(1) During the measurement of the impedance, the muscle of the thighs of the subject should be in the bending state close to the bending state of the muscle of pretest subjects when the estimation formulae were prepared; that is, with the knees straightened or almost straightened.

(2) The bending angle of the knees of the subject should be fixed during the measurement.

(3) The contact positions of the electrodes should be accurately determined; the positioning of the electrodes should be reproducible.

If the accuracy of determining the measurement points (i.e. voltage-measuring points described above) is low, it is difficult to perform the measurement with high reproducibility. The reason is explained as follows. From the cross-sectional area (A), length (L) and volume resistivity (,,) of a segment, the impedance (Z) of the segment is calculated as follows:

Z=,,×(L/A).

Therefore, the volume (V) is given as follows:

V=A×L=(,,×L/Z)×L=,,×(L²/Z).

From this formula, it is obvious that the volume of a muscle is proportional to the second power of the length of the target part. This means that the accuracy of determination of measurement points greatly influences the measurement result.

Another important condition is that the subject should not be forced to take an unnatural position. Also, it is desirable that even aged people, children or those who lack flexibility of the body can easily perform the measurement. To satisfy these conditions, the apparatuses according to the first to fourth embodiments, to be described below, are constructed so that the knees of the subject are maintained at a bending angle within the range from about 140 to 180 degrees when the subject sits down on the floor or lies on the floor in a supine position, and so that the impedance is measured mainly with electrodes arranged to contact the backs of the knees.

[First Embodiment]

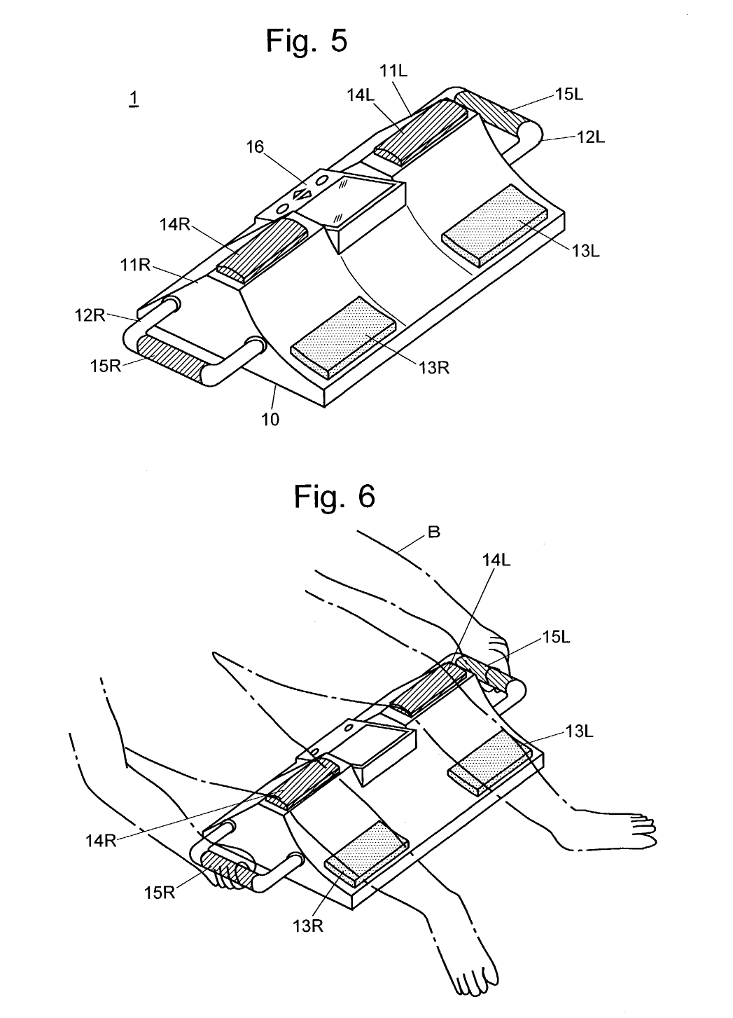

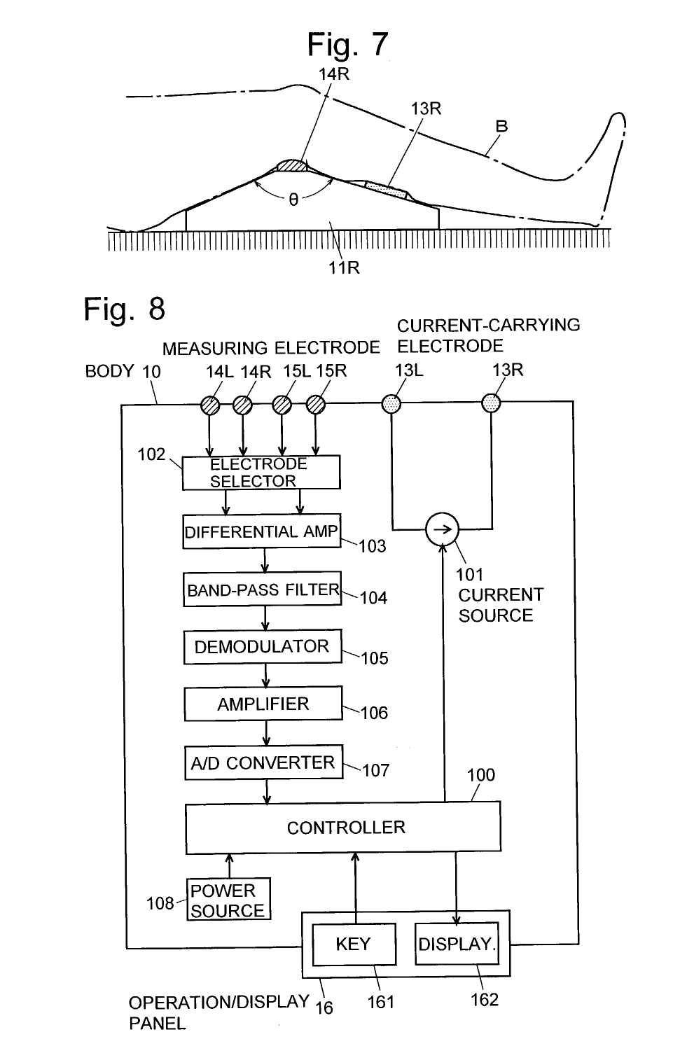

FIG. 5 is a perspective view of a body composition measurement apparatus 1 according to the first embodiment of the present invention; FIG. 6 is a perspective view of the measurement apparatus 1 being used in a measurement; and FIG. 7 is a side view of the apparatus 1 being used. This measurement apparatus 1 has a body 10 having tapered parts 11L and 11R formed like slopes. The tapered parts 11L and 11R are designed so that the bending angle ,, of the knees of the subject B becomes about 160 degrees when the subject B sits down on the floor with the legs laid over the tapered parts 11L and 11R. At the top of the tapered parts 11L and 11R, measuring electrodes 14L and 14R are arranged to contact the backs of the knees. On the slopes of the tapered parts 11L and 11R, current-carrying electrodes 13L and 13R are arranged to contact the backs of the calves. U-shaped grip bars 12L and 12R are connected to both sides of the body 10. The grip bars 12L and 12R have measuring electrodes 15L and 15R, respectively, which contact the palms of the subject B when gripped with both hands. At the top of the body 10, an operation/display panel 16 having operation keys and a display device is provided between the tapered parts 11L and 11R. This panel 16 is inclined for better visibility from the subject B.

The current-carrying electrodes and the measuring electrodes may be made of stainless steel or other metals. To ensure the contact, however, these electrodes may be made of conductive rubber or other cushioning materials, or conductive plastic. Alternatively, the electrode may include a cushioning medium provided with a metallic film or other conductive material on its contact face.

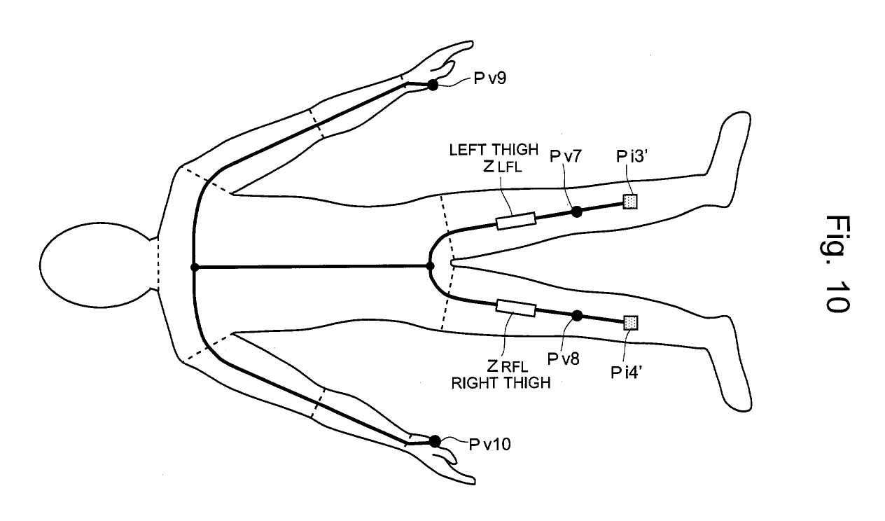

When the subject B takes a proper measuring position as shown in FIG. 6, the current-carrying electrodes 13L and 13R contact the backs of both calves set apart by a preset distance, the measuring electrodes 14L and 14R contact the backs of both knees, and the measuring electrodes 15L and 15R contact both palms. This measuring position provides two current-supplying points Pi3′ and Pi4′ and four voltage-measuring points Pv7, Pv8, Pv9 and Pv10, as shown in FIG. 10. The current-supplying points Pi3′ and Pi4′ correspond to the current-supplying points Pi3 and Pi4 in FIG. 1. The current-supplying points may be determined as desired as long as these points are located farther from the trunk than the voltage-measuring points Pv7 and Pv8, and as long as these points are set apart from the voltage-measuring points Pv7 and Pv8 by a distance greater than the minimum distance for satisfying the condition for impedance measurement according to the four-electrode method.

FIG. 8 shows the configuration of the electrical system of the apparatus 1. Current-carrying electrodes 13L and 13R are connected to a current source 101, which generates a constant-current radio-frequency signal at frequency f0. Usually, the frequency f0 of the radio-frequency signal is determined appropriately within the range from 10 kHz to 100 kHz. Measuring electrodes 14L, 14R, 15L and 15R are connected to an electrode selector 102. According to the instruction from a controller 100, the electrode selector 102 selects two measuring electrodes and connects them to the input of a differential amplifier 103.

The output of the differential amplifier 103 is connected to a band-pass filter (BPF) 104, which filters out signal components other than the frequency f0. After that, a demodulator 105 performs demodulation and rectification to extract the signal component of frequency f0, which is then amplified by an amplifier 106. The signal is converted into digital signals by an analog-to-digital (A/D) converter 107, which sends the digital signal to the controller 100. The controller 100 is composed of a microcomputer or microcomputers and other devices; the microcomputer includes CPU, ROM and RAM. According to the control program preinstalled in the ROM, the controller 100 performs various operations, including measurement of impedance, estimation and calculation of body composition information, etc. In addition, the body 10 includes a power source 108, such as a battery.

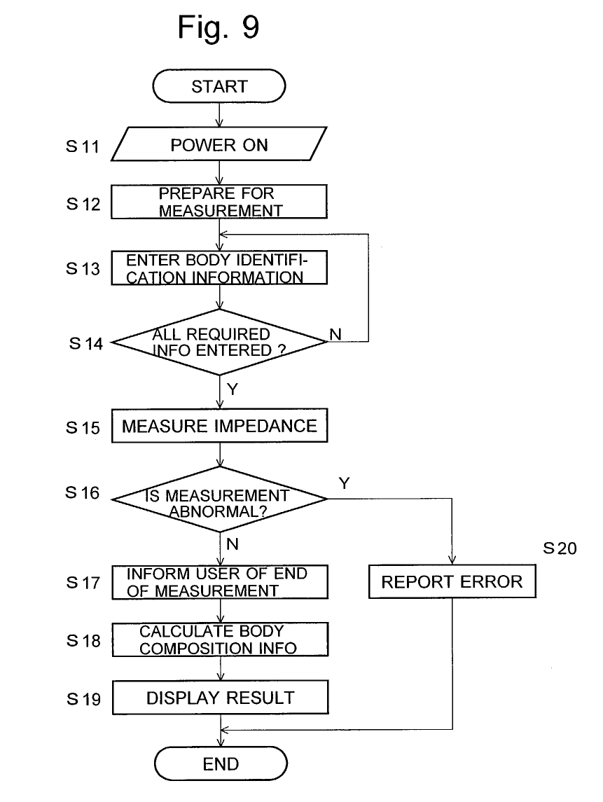

The process of measuring body composition using the measurement apparatus 1 is described below, referring to the flowchart shown in FIG. 9. First, the subject B lays both legs on the body 10, as shown in FIG. 6, and presses a power switch provided on the operation/display panel 16 to energize the apparatus 1 (Step S11). Then, the apparatus 1 starts running, and performs initialization, self-inspection of the measurement circuit, and other processing to prepare for the measurement (Step S12). Next, the subject B enters the height, age, sexuality and other body identification information by using the operation key 161 in a predetermined manner (Step S13). Then, the controller 100 determines whether the minimally required items of information have been entered (Step S14). If any required item is missing, the process returns to Step S13, where the subject B is prompted to enter the missing information. When all the required items have been entered, the controller 100 conducts the impedance measurement (Step S15).

In this measurement, a weak radio-frequency current is supplied from the power source 101 between the current-carrying electrodes 13L and 13R. This generates a flow of current passing through the left thigh and the right thigh. With the current thus flowing, the electrode selector 102 selects the measuring electrode 14L, which is in contact with the left knee, and the measuring electrode 15L, which is in contact with the left palm. In this state, the potential difference between these electrodes is measured, and the measurement value is sent to the controller 100. As is clear from FIG. 10, the voltage measured thereby must be equal to the voltage across the impedance Z_{LFL} of the left thigh. Therefore, the impedance Z_{LFL} of the left thigh can be calculated from the above voltage.

In the above measurement, both palms are used as the voltage-measuring points. It is also possible to use the palm as the voltage-supplying point. As can be understood from FIG. 10, if the current is supplied between the point Pv9 (left palm) and the point Pi3′, the potential difference between the points Pv7 and Pv8 reflects the impedance Z_{LFL} of the left thigh. Thus, the impedances of both thighs, Z_{LFL} and Z_{RFL}, can be separately measured by using the measuring electrodes 15L and 15R to supply the current.

Next, the electrode selector 102 selects the measuring electrode 14L, which is in contact with the left knee, and the measuring electrode 15R, which is in contact with the right palm. In this state, the potential difference between these electrodes is measured, and the measurement value is sent to the controller 100. In this case, the voltage measured hereby must be equal to the voltage across the impedance Z_{LFL} of the left thigh, as in the previous case. However, if the modeling of the trunk and both arms as lead wires is unbalanced between the right and left sides of the body, a slight discrepancy arises in the voltage measured between the two cases. Taking this into account, the average of the measurement results of the two cases may be adopted as the impedance Z_{LFL} of the left thigh, whereby the accuracy of measurement is improved.

Next, the electrode selector 102 selects the measuring electrode 14R, which is in contact with the right knee, and the measuring electrode 15R, which is in contact with the right palm. In this state, the potential difference between these electrodes is measured, and the measurement value is sent to the controller 100. After that, the electrode selector 102 selects the measuring electrode 14L, which is in contact with the right knee, and the measuring electrode 15L, which is in contact with the left palm. Again, in this state, the potential difference between these electrodes is measured, and the measurement value is sent to the controller 100. Thus, similar to the above case, the impedance Z_{RFL} of the right thigh can be obtained with high accuracy.

If the voltage is abnormally high or low, or if the measurements performed plural times on the same part of the body produce a diversity of results, the measurement is probably incorrect (because of inadequate contact of the electrodes, for example). In such a case, the measurement is determined as abnormal (“Yes” in Step S16). Then, the error is reported with a display or buzzer (Step S20), and the measurement is terminated.

When all the measurements are determined as normal, the subject B is informed of the completion of the measurement by, for example, a message on a display device 162 (Step S17). With this message, the subject B is allowed to release herself or himself from the measuring position, that is, to lift the legs off the body 10. After that, the controller 100 performs predetermined operations based on the measurement value of the impedance and the body identification information entered beforehand, to produce body composition information and health check information (Step S18), and displays the result on the display device 162 (Step S19). For example, the muscle masses of both thighs are estimated, and the muscle masses are displayed with the state of balance between the right and left sides of the body. It is of course possible to estimate and display other kinds of information, as described above.

As explained above, with the apparatus according to the first embodiment, the subject B can measure and obtain various kinds of information about body composition and/or health condition without difficulty and in a comfortable position.

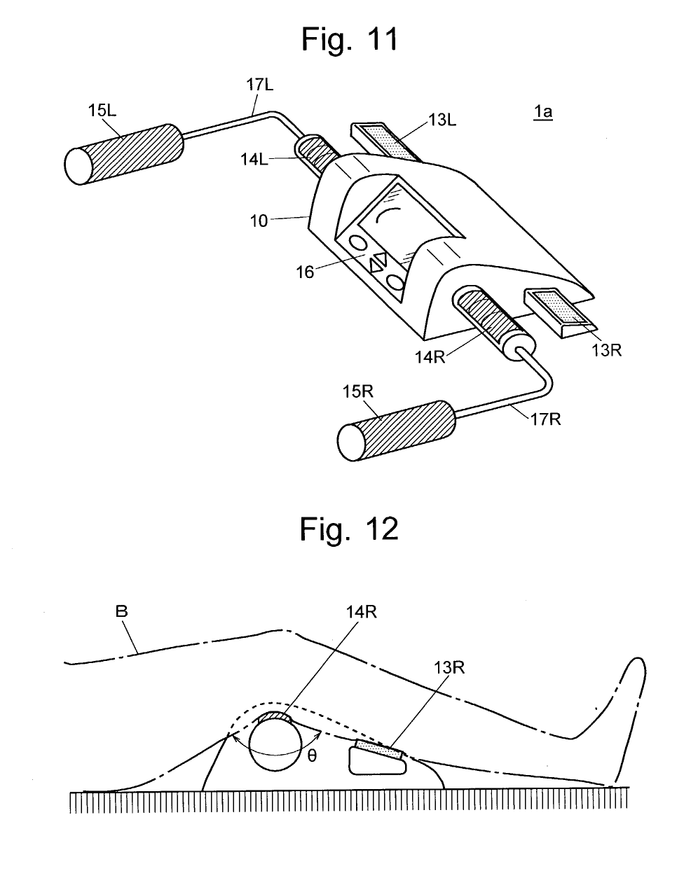

FIG. 11 is a perspective view of a body composition measurement apparatus la as a variation of the first embodiment, and FIG. 12 is a side view of the measurement apparatus 1a being used in a measurement. It should be noted that any component of the apparatus 1a identical or corresponding to a component mentioned in the description of the first embodiment will be denoted by the same numeral, and will not be described in detail unless it is necessary. This rule also applies to other embodiments to follow.

This apparatus la has L-shaped bars 17L and 17R extending from both sides of the body 10 containing electrical circuits. The bars 17L and 17R have a pair of columnar measuring electrodes 14L and 14R at their roots, and another pair of columnar measuring electrodes 15L and 15R at their ends. Current-carrying electrodes 13L and 13R are also provided on both sides of the body 10. The electrodes are positioned so that when, as shown in FIG. 12, the subject lays the knees on the measuring electrodes 14L and 14R and the calves on the current-carrying electrodes 13L and 13R, the bending angle ,, of the knees becomes about 160 degrees. The body 10 is designed to be clamped by both legs. This design ensures both legs to be apart from each other by a predetermined distance, equal to the width of the body 10, so that both thighs are prevented from contacting each other.

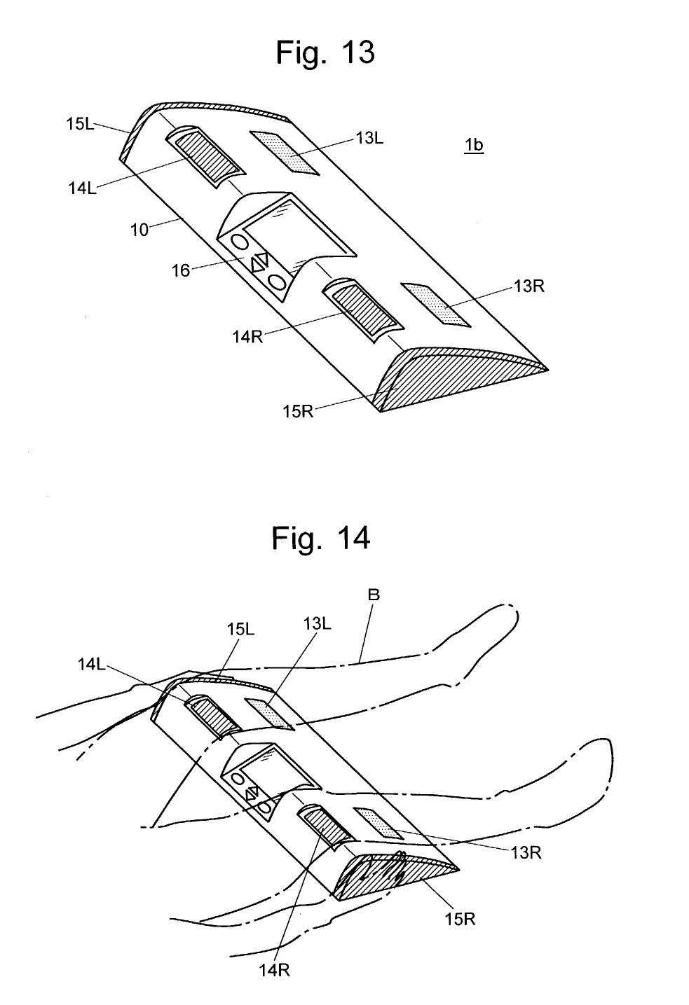

FIG. 13 is a perspective view of a body composition measurement apparatus 1b as another variation of the first embodiment, and FIG. 14 is a perspective view of the measurement apparatus 1b being used in a measurement. In this apparatus 1b, the measuring electrodes 15L and 15R are located on both sides of the body 10 containing electrical circuits. To use this apparatus 1b, the subject B touches the measuring electrodes 15L and 15R with both palms, as if holding the body 10 with both hands, as shown in FIG. 14. It is of course possible to form a projection or similar structure on both sides of the body 10 and provide the measuring electrodes 15L and 15R there so that the subject can easily grip or hold the measuring electrodes 15L and 15R.

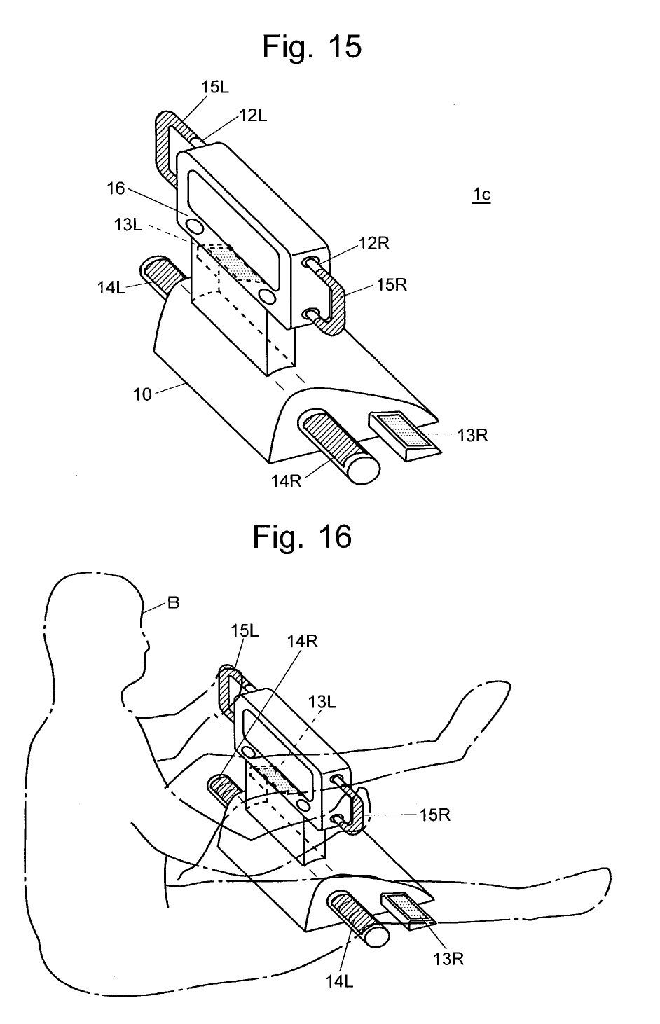

FIG. 15 is a perspective view of a body composition measurement apparatus 1c as another variation of the first embodiment, and FIG. 16 is a perspective view of the measurement apparatus 1c being used in a measurement. In this measurement apparatus 1c, the operation/display panel 16 stands on the top of the body 10 containing electrical circuits, and the measuring electrodes 15L and 15R are provided on a pair of U-shaped grip bars 12L and 12R projecting from both sides of the operation/display panel 16. This construction allows the subject B to grip the bar 12L and 12R in a comfortable position, so that the palms assuredly contact the measuring electrodes 15L and 15R.

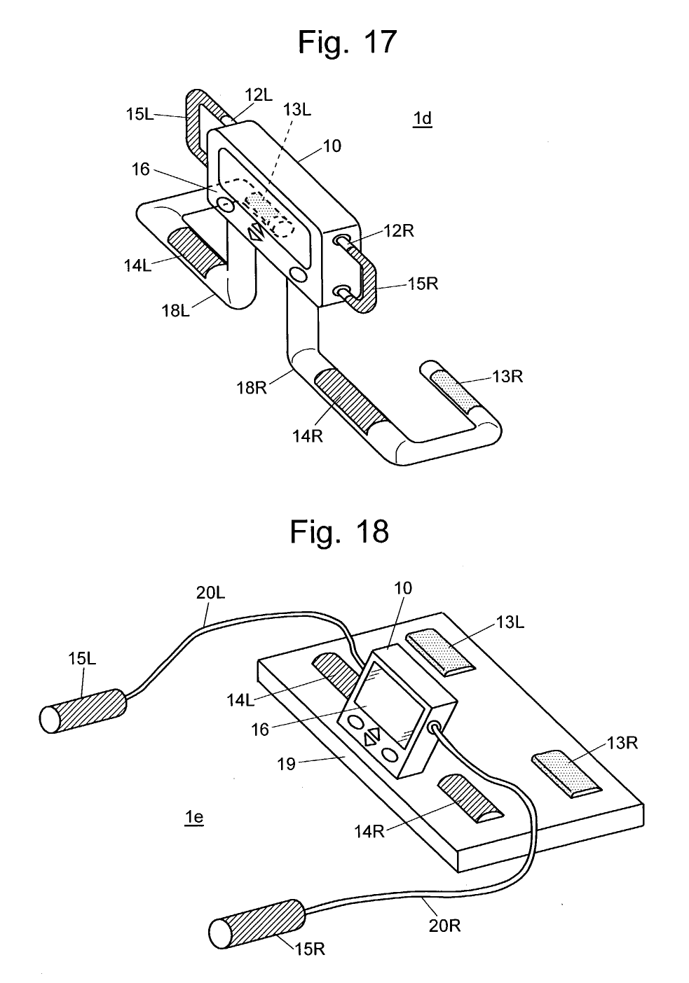

FIG. 17 is a perspective view of a body composition measurement apparatus 1d as another variation of the first embodiment. In this measurement apparatus 1d, a pair of supporting legs 18L and 18R extends from the bottom of the body 10 having the operation/display panel 16 on its front side. The measuring electrodes 14L and 14R and the current-carrying electrodes 15L and 15R are provided on the supporting legs 18L and 18R.

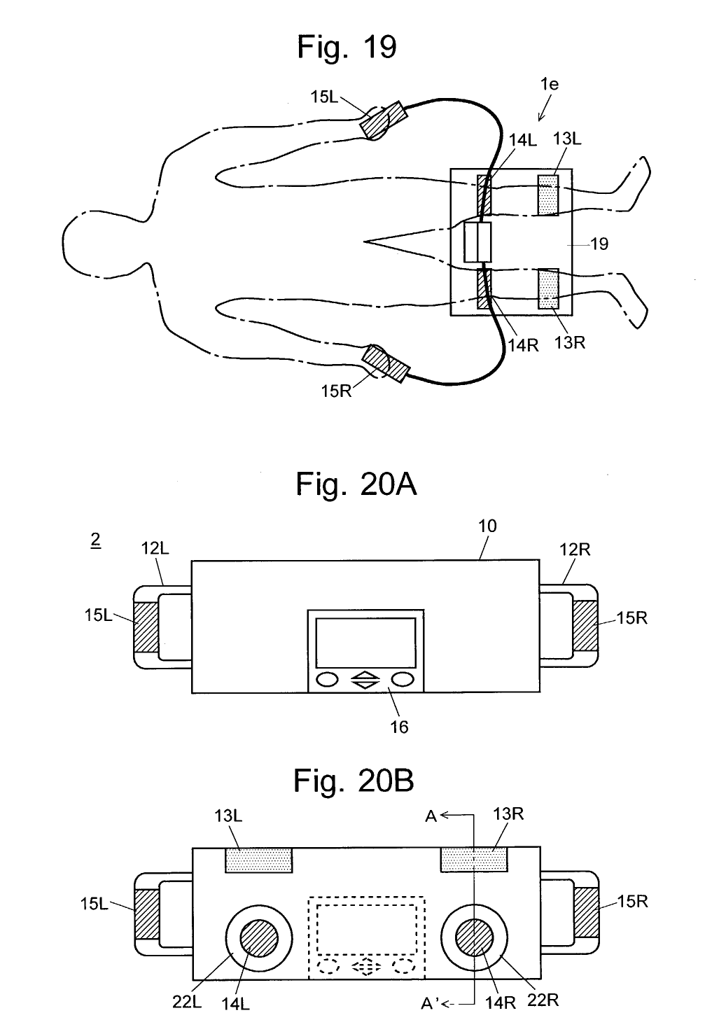

FIG. 18 is a perspective view of a body composition measurement apparatus 1e as another variation of the first embodiment, and FIG. 19 is a top view of the measurement apparatus 1e being used in a measurement. In this measurement apparatus 1e, the body 10 includes a platform 19 on which the measuring electrodes 14L and 14R, current-carrying electrodes 13L and 13R and operation/display panel 16 are arranged, and grip-like measuring electrodes 15L and 15R, to be held by hands, are connected to the body 10 with cables 20L and 20R. With this measurement apparatus 1e, the subject B can perform the measurement in a sitting position with both legs stretched out. Alternatively, it is possible to take a comfortable supine position, as shown in FIG. 19.

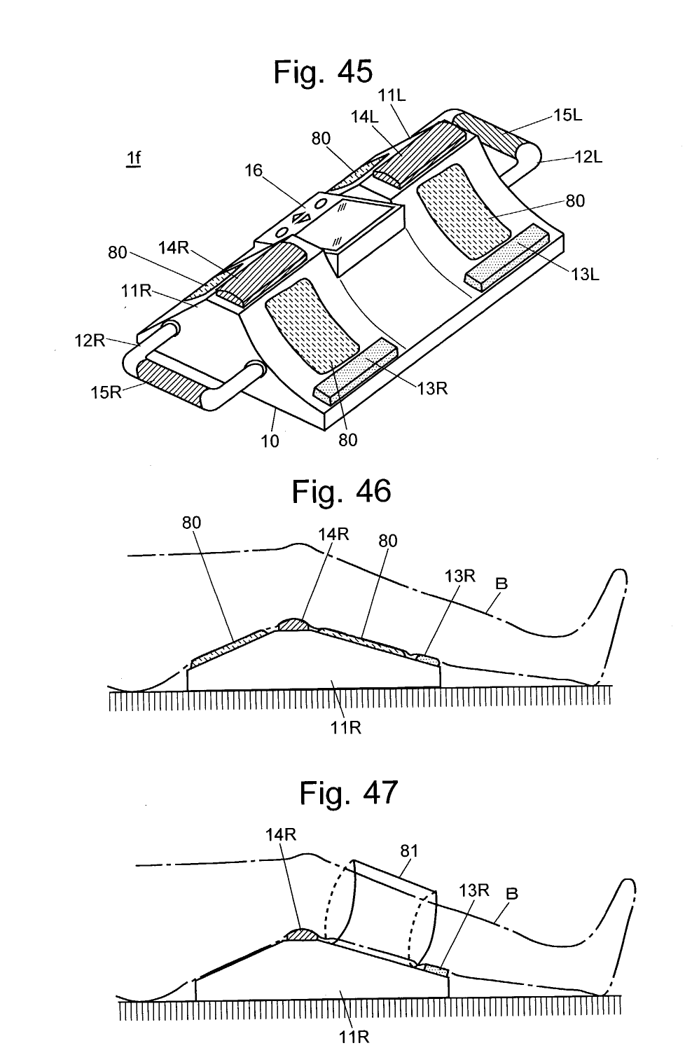

The body composition measurement apparatus according to the first embodiment, and its variations described above, may further include an apparatus for providing desirable effects on the tissue of the entire body or a part of the body. FIG. 45 is a perspective view of a measurement apparatus If as an example of such apparatus, and FIG. 46 is a side view of the measurement apparatus If being used in a measurement.

In this example, infrared heaters 80 are provided on the tapered parts 11L and 11R of the body 10. When the subject B lays the legs on the tapered parts 11L and 11R, as shown in FIG. 46, the infrared heaters 80 contact the backs of the calves and thighs of the subject B. The heaters 80 provide a massaging effect on the body of the subject B by warming the legs. Thus, using the measurement apparatus If, the subject B can have the lower limbs thermally massaged any time. It is also possible to check the effects of the massage on the promotion of blood circulation or the elimination of swelling by measuring the body composition as described above before and after the massage.

In addition to the thermal type of stimulating apparatus, it is possible to include other types of apparatuses that provide favorable effects on the living body tissue by, for example, mechanically or electrically stimulating the tissue. FIG. 47 shows an example where air massagers 81 for wrapping the calves of the subject B are provided on the tapered parts 11L and 11R.

[Second Embodiment]

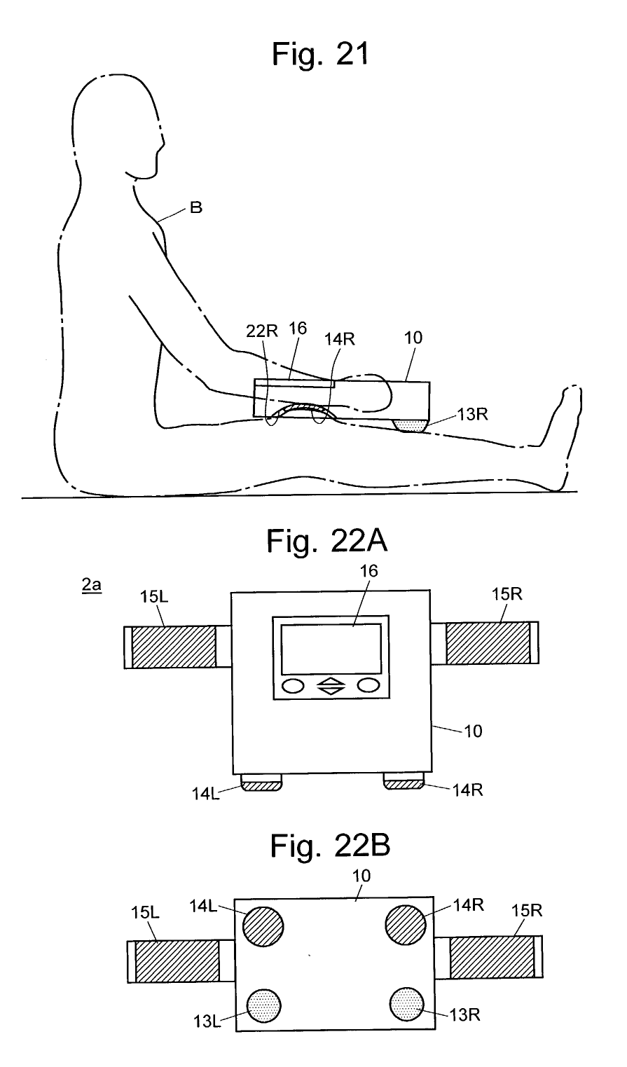

FIGS. 20A-20B show a body composition measurement apparatus 2 according to the second embodiment of the present invention, where FIG. 20A is a top view, and FIG. 20B is a bottom view. FIG. 21 is a side view of the measurement apparatus 2 being used in a measurement. In FIG. 21, the measurement apparatus 2 is depicted by a cross-sectional drawing at line A-A′ in FIG. 20B. In the first embodiment and its variations, the measuring electrodes 14L and 14R are arranged to contact backs of knees. In the second embodiment, on the other hand, these electrodes are arranged to contact the fronts of the knees (i.e. kneecaps).

The square-shaped flat body 10 has the following elements: operation/display panel 16 on its top; U-shaped grip bars 12L and 12R with the measuring electrodes 15L and 15R on its both sides; measurement electrodes 14L and 14R and current-carrying electrodes 13L and 13R on its bottom side, where two electrodes constituting each pair are spaced by a preset distance. The measuring electrodes 14L and 14R are located inside the cup-shaped hollows 22L and 22R. To use this measurement apparatus, the subject sits down with the legs stretched out, places the apparatus 2 on the legs, and holds the measuring electrodes 15L and 15R with both hands, as shown in FIG. 21. There, the bulges of the kneecaps of the subject B enter the hollows 22L and 22R, so that the measuring electrodes 14L and 14R assuredly contact the tops of the kneecaps, while the current-carrying electrodes 13L and 13R contact the front side of the shanks. Thus, two current-supplying points and four voltage-measuring points are defined as in the first embodiment, so that the measurement can be similarly performed.

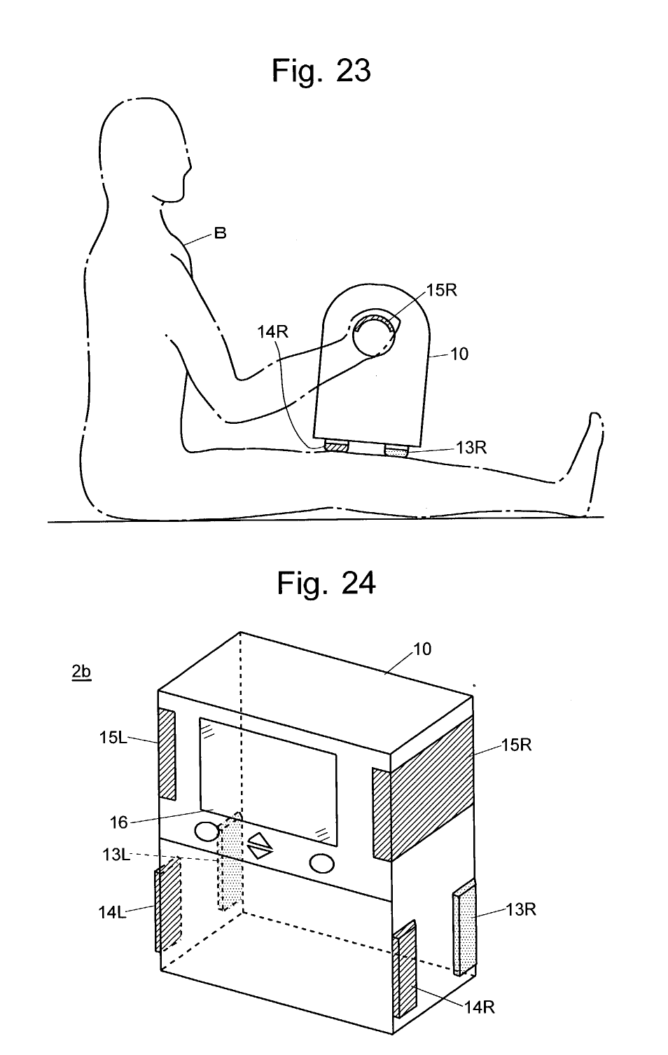

FIGS. 22A-22B shows a body composition measurement apparatus 2a as a variation of the second embodiment, where FIG. 22A is a front view, and FIG. 22B is a bottom view. FIG. 23 is a side view of the measurement apparatus 2a being used in a measurement. In the measurement apparatus 2a, the body 10 has the following elements: operation/display panel 16 on its front side; measuring electrodes 15L and 15R in the form of a columnar grip projecting from both sides; measuring electrodes 14L and 14R and current-carrying electrodes 13L and 13R projecting from its bottom side. Also in this case, the subject B places the apparatus 2a on the legs to perform the measurement.

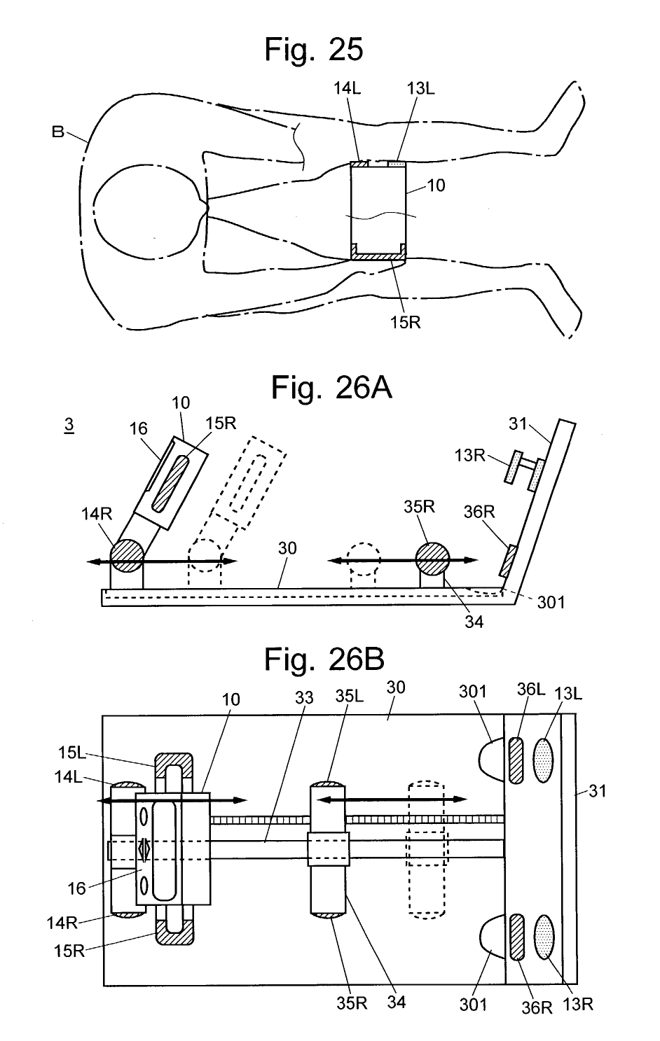

FIG. 24 is a perspective view of a body composition measuring apparatus 2b as another variation of the second embodiment, and FIG. 25 is a top view of the measurement apparatus 2b being used in a measurement. In the measurement apparatus 2b, the square-shaped body 10 has the following elements: operation/display panel 16 on its front side; measuring electrodes 15L and 15R on the upper part of both sides; measurement electrodes 14L and 14R and current-carrying electrodes 13L and 13R on the lower part of both sides, where the two electrodes located on each side are spaced by a preset distance. To use this apparatus 2b, the subject B sits down with the legs stretched out, clamps the body 10 with the legs so that the insides of the knees contact the measuring electrodes 14L and 14R, and holds the upper part of the body 10 from both sides with both hands, as shown in FIG. 25. Here, the measuring electrodes 14L and 14R are in contact with the insides of the knees of the subject B, and the current-carrying electrodes 13L and 13R are in contact with the insides of the shanks or calves. Thus, the two current-supplying points and the four voltage-measuring points are defined as in the first embodiment.

[Third Embodiment]

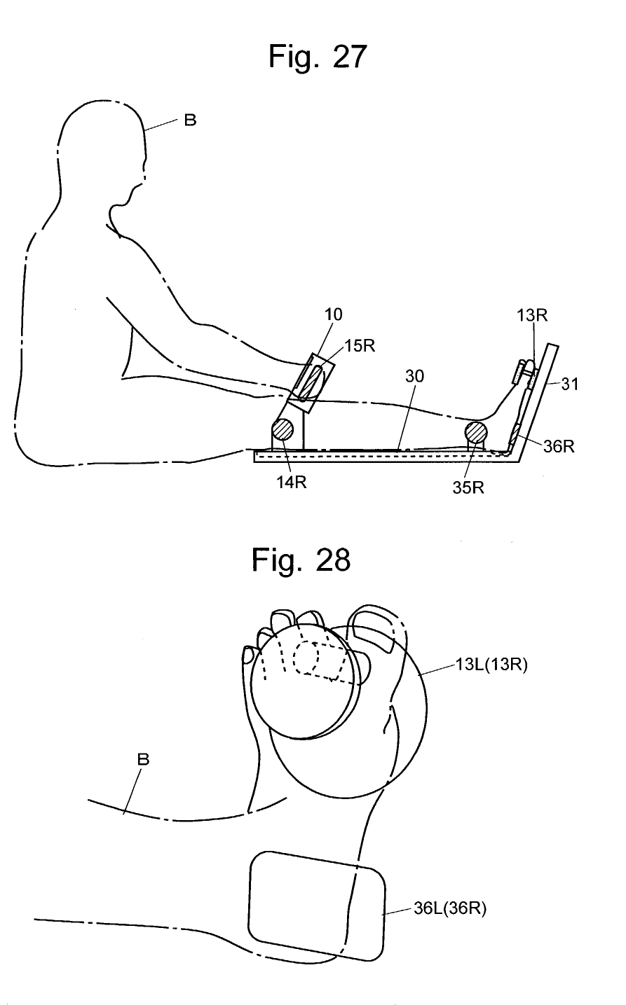

FIGS. 26A-26B shows a body composition measurement apparatus 3 according to the third embodiment of the present invention, where FIG. 26A is a side view and FIG. 26B is a top view. FIG. 27 is a side view of the measurement apparatus 3 being used in a measurement, and FIG. 28 is an enlarged perspective view of a foot under measurement. Different from the apparatuses according to the first and second embodiments and their variations, the measurement apparatus 3 is constructed to measure the impedances of calves and ankles in addition to the impedances of thighs.

The measurement apparatus 3 has a standing plate 31 at an end of a horizontal base 30. The standing plate 31 has current-carrying electrodes 13L and 13R to be inserted between the first and second fingers of the feet of the subject B, and measuring electrodes 36L and 36R arranged to contact the heels of the subject B. On the horizontal base 30, a body 10 having measuring electrodes 14L and 14R on both sides of its lower part is slidably placed on the rail 33. Also, an electrode holder 34 is slidably placed on the rail 33 between the body 10 and the standing plate 31. The electrode holder 34 has measuring electrodes 35L and 35R on both sides, which are arranged to contact the insides of both ankles.

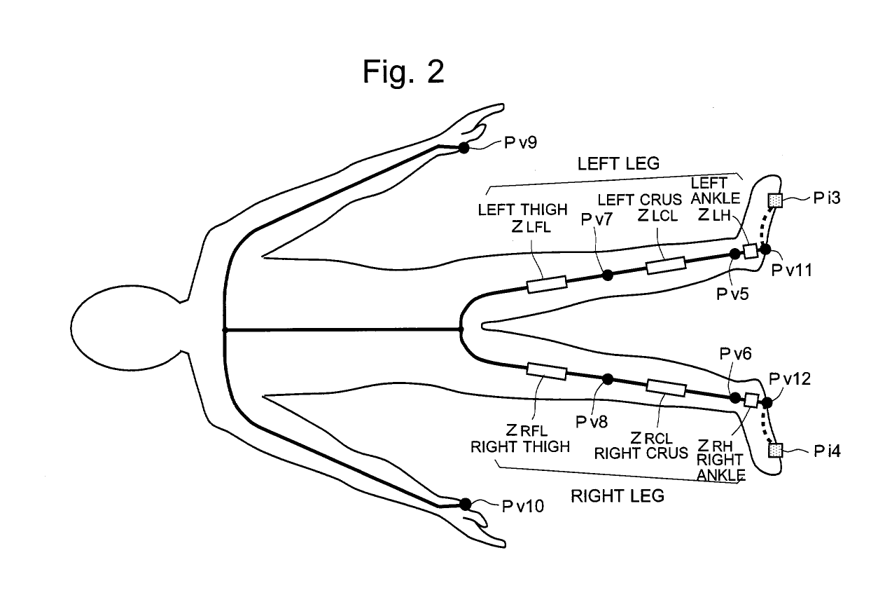

To use this apparatus 3, the subject B sits down with the legs stretched out on both sides of the rail 33 so that the soles of the feet contact the standing plate 31 with the current-carrying electrodes 13L and 13R inserted between the first and second fingers of the feet. Then, the positions of the body 10 and the electrode holder 34 along the rail 33 are adjusted so that the measuring electrodes 14L and 14R contact the insides of the knees, and the measuring electrodes 35L and 35R contact the insides of the ankles. This positioning provides two current-supplying points Pi3 and Pi4 and six voltage-measuring points Pv7, Pv8, Pv5, Pv6, Pv11 and Pv12 as shown in FIG. 2. The measuring electrodes 14L, 14R, 35L and 35R can assuredly contact the knees and the ankles, irrespective of the body size of the subject, because the positions of the measuring electrodes 14L, 14R, 35L and 35R can be changed as desired according to the positions of the knees and the ankles of the subject B.

Furthermore, in the measurement apparatus 3, the body 10 and the electrode holder 34 have built-in range finders or position sensors, respectively. These sensors are arranged to measure the distance between the measuring electrode 14L (or 14R) and the measuring electrodes 35L (or 35R) and the distance between measuring electrodes 35L (or 35R) and the measuring electrode 36L (or 36R). Any type of sensor can be used here, as long as it can measure the distance between two objects. For example, sensors using ultrasonic waves or light, or mechanical sensors may be used. In any case, the distance measured by the sensors corresponds to the body size of the subject B. Therefore, the distance can be used as the information relating to the length of the target part of the body.

In general, the length of a body part, such as length of thigh, is highly correlated with the height and can be estimated from the height entered as one item of body identification information. By the measurement apparatus 3 according to the third embodiment, on the other hand, the length of the body part is not estimated but actually measured with high accuracy. Therefore, the measurement accuracy is greatly improved.

In the measurement apparatus 3, the number of voltage-measuring points has increased from four to six. Accordingly it is possible to independently measure the impedances of the thighs (Z_{LFL}, Z_{RFL}), the impedances of the calves (Z_{LCL}, Z_{RCL}) or the impedance of the ankles (Z_{LH}, Z_{RH}) by appropriately selecting the voltage-measuring points. Therefore, with the measurement apparatus 3, the subject can obtain other kinds of body composition information, such as the muscle mass, bone mass and bone density of each body part, which cannot be obtained with the apparatus according to the first or second embodiment.

For example, the ankle has such thin subcutaneous fat layer and muscular tissue layer that the ratio of bone is rather large compared to the ratio of muscle or fat. This means that, in the case of the model shown in FIG. 4, the cross-sectional area of the bone is rather great. Accordingly, when the voltage between the bottom of the heel and the ankle is measured with a radio-frequency current being supplied between both legs, the impedance calculated from the radio-frequency current and the voltage measured contains information about the osseous tissue at a part around the ankle. Therefore, using the impedance thus measured, it is possible not only to calculate the bone mass of the body part concerned, but also to improve the accuracy of estimation of the total bone mass. Furthermore, based on detailed information about bone tissue, it is possible to obtain information about bone density, progress of osteoporosis, etc., as information indicating the health conditions of the bone.

[Fourth Embodiment]

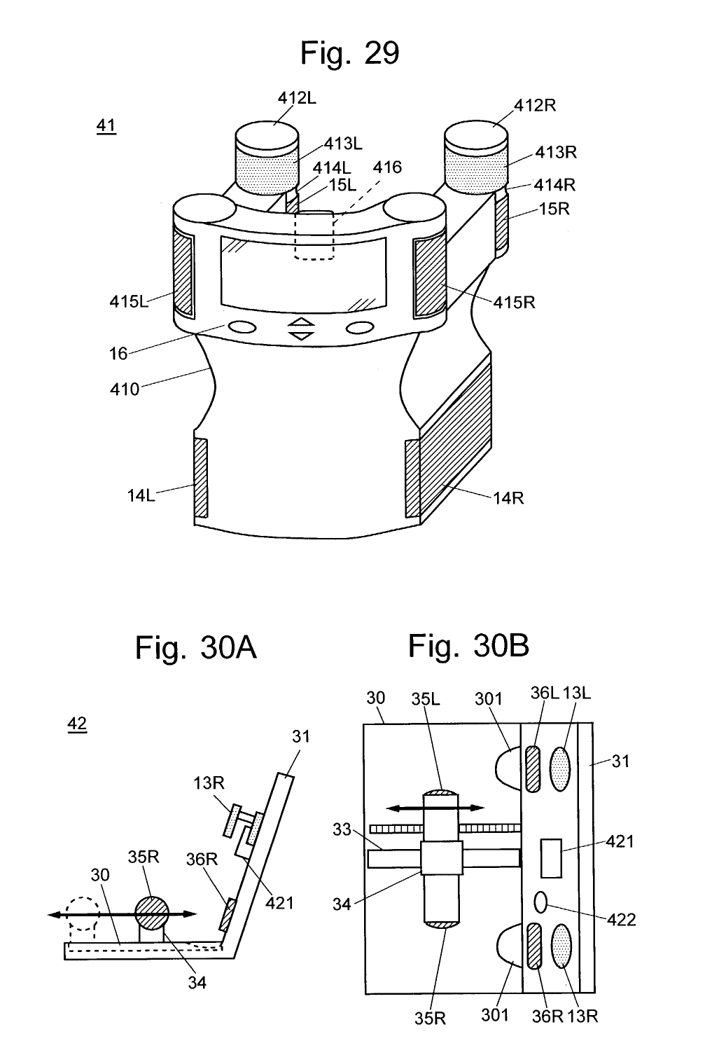

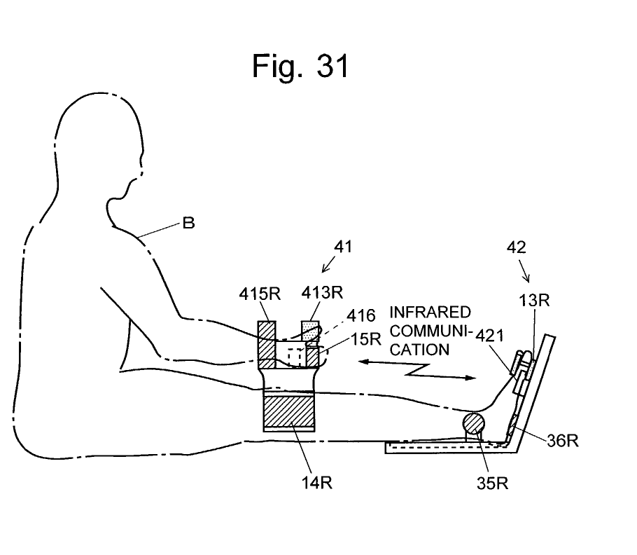

The body composition measurement apparatus according to the fourth embodiment includes a first measurement unit for mainly measuring upper limbs and a second measurement unit for mainly measuring lower limbs. FIG. 29 is a perspective view of the first measurement unit 41 of the body composition measurement apparatus 4 according to the fourth embodiment. FIGS. 30A-30B show the second measurement unit 42, where FIG. 30A is a side view and FIG. 30B is a top view. FIG. 31 is a side view of the measurement apparatus 4 being used in a measurement. The second measurement unit 42 is basically composed of the same elements as used in the measurement apparatus 3 at the part used for measuring legs lower than the knees. The standing plate 31 has an infrared communication module 421 and an indicator 422. The communication module 421 is used for infrared communication, which will be described later, and the indicator 422 is used for indicating the state of the infrared communication.

The first measurement unit 41 has a body 410 that is U-shaped when viewed from the top, where both ends of the U-shaped body are directed backwards. At both ends of the body 410, columnar grips 412L and 412R are provided. On the circumferential side faces of the grips 412L and 412R, current-carrying electrodes 413L and 413R are provided in the upper parts and measuring electrodes 15L and 15R are provided in the lower parts, with a gap between them. At the bending parts of the body 410, other measuring electrodes 415L and 415R are provided on the outer side. The operation/display panel 16 is located on the front side of the body 410 between the measuring electrodes 415L and 415R. In the lower part of the body 410, measuring electrodes 14L and 14R are provided on both sides. An infrared communication module is provided on the back side of the body 410.

To use this apparatus 4, the subject B holds the grips 412L and 412R with both hands, with the arms stretched out, where both thumbs are put on the front sides of the grips 412L and 412R, and other fingers are put on the back sides. There, the entirety of both thumbs and the finger cushions of the index and middle fingers contact the current-carrying electrodes 413L and 413R, both palms contact the measuring electrodes 15L and 15R, and the insides of both wrists contact the measuring electrodes 415L and 415R. This measuring position provides two current-supplying points Pi1 and Pi2 and four voltage-measuring points Pv1, Pv2, Pv9 and Pv10. The current-carrying electrode 413L (413R) and the measuring electrode 15L (15R) can exchange their functionalities without causing essential change in performance.

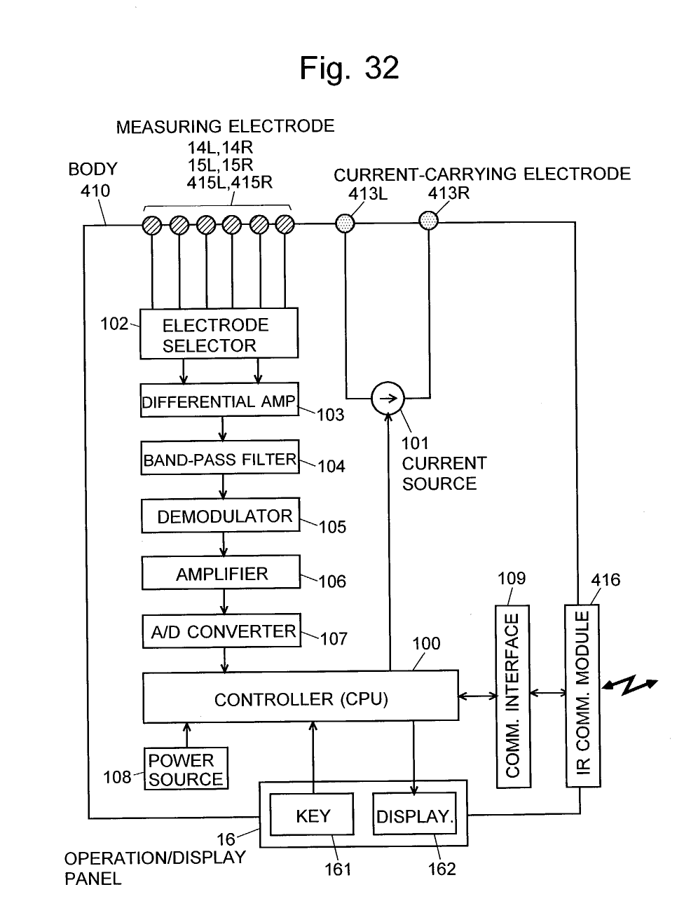

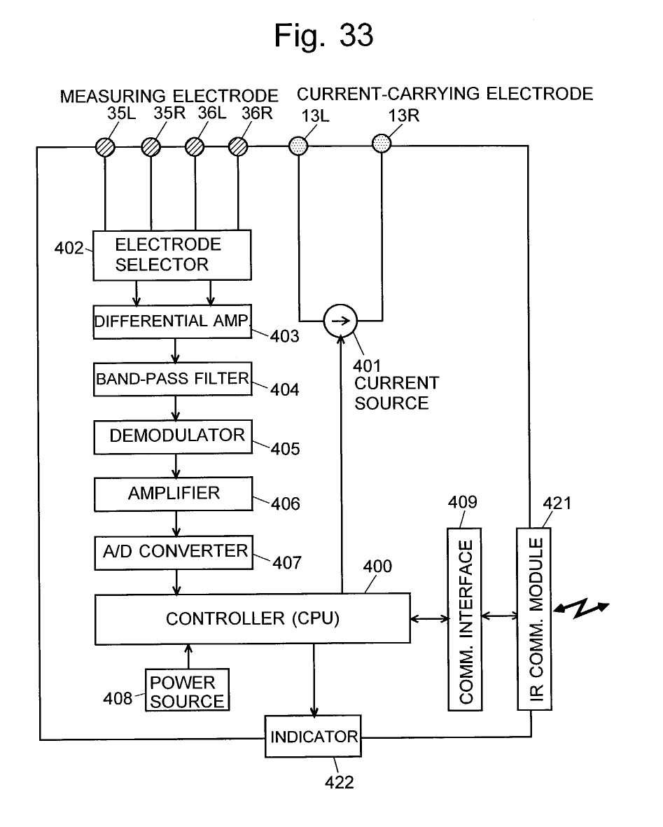

FIG. 32 shows the configuration of the electrical system of the first measurement unit 41, and FIG. 33 shows the configuration of the electrical system of the second measurement unit 42. The measurement units 41 and 42 have controllers 100 and 400, respectively. The controller 100 in the first measurement unit 41 serves as a master, which has every function for estimating body composition information. The controller 400 in the second measurement unit 42, on the other hand, serves as a slave, which conducts only the measurement of impedance according to the instructions from the master.

After the measurement is started, the first measurement unit 41 sends signals indicating the start of measurement or other timing concerning measurement control to the measurement unit 42 according to necessity. In response to the signal, the second measurement unit 42 conducts a measurement using the measuring electrodes 35L, 35R, 36L and 36R, and sends the information obtained thereby to the first measurement unit 41 as digital data through the infrared communication between the infrared communication modules 416 and 421. From the information transferred from the second measurement unit 42, the first measurement unit 41 can calculate the potential difference between the voltage-measuring points located lower than the knees, and measure the impedance of the crus or the ankle. In addition, the first measurement unit 41 itself can conduct the measurement of impedance using the measuring electrodes 14L, 14R, 15L, 15R, 415L and 415R. Therefore, it is possible to measure not only the impedance of the lower limbs but also the impedance of the upper limbs and the trunk. Thus, the measurement apparatus 4 can provide more detailed and accurate information than the other apparatuses described above.

When, as in the above embodiment, the measurement apparatus according to the present invention is constructed from plural units, the communication between the units may be achieved by using infrared or other kinds of light, or by other wireless communication method using radio waves, ultrasonic waves, etc. It is of course possible to adopt wire communication methods using cables.

In the above-described embodiments and their variations, the subject B sits down on the floor or similar place, stretching the legs out with the knees appropriately bent. While this positioning is preferable with respect to the accuracy of measurement, the subject may feel physically burdened or forced, depending on the health condition. Taking this into account, in the following embodiments, the measurement apparatuses are constructed so that the subject can take more comfortable positions during the measurement. That is, the following measurement apparatuses are constructed so that the subject is allowed to sit down on a chair-like body, with both knees bent to the angle of about 90 degrees.

[Fifth Embodiment]

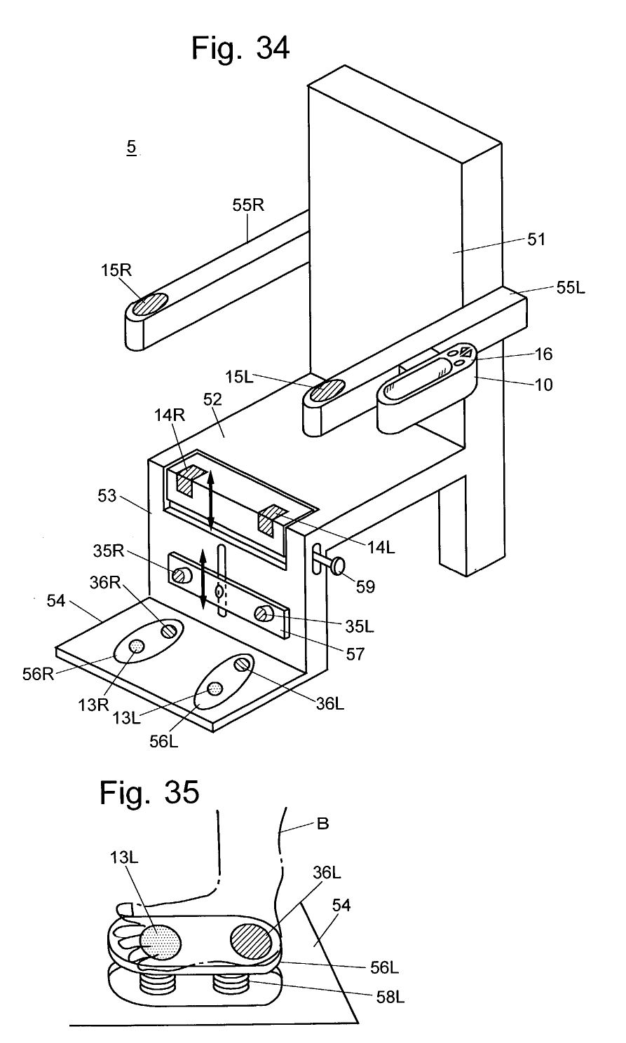

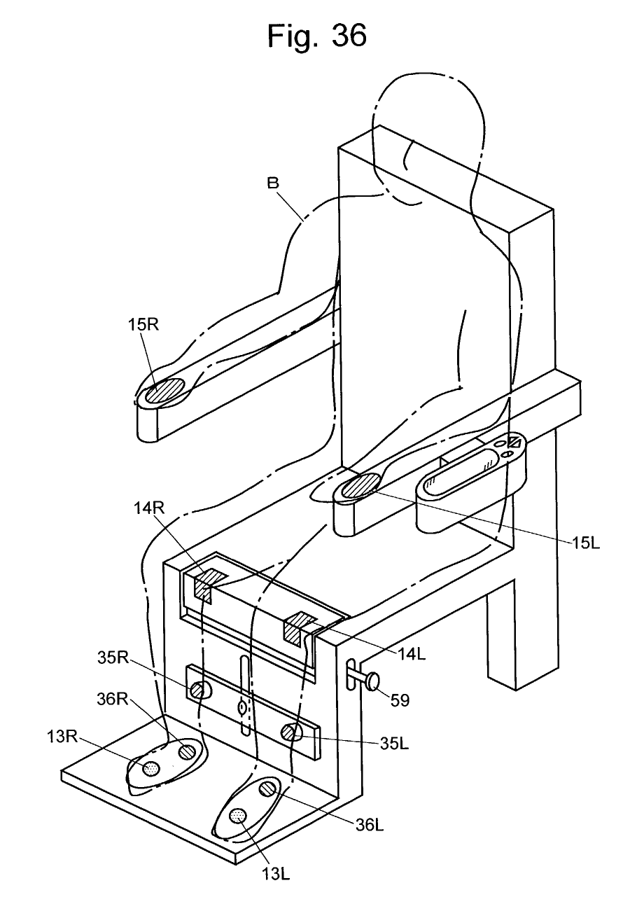

FIG. 34 is a perspective view of a body composition measuring apparatus 5 according to the fifth embodiment of the present invention, FIG. 35 is a perspective view of a part of the measurement apparatus 5, and FIG. 36 is a perspective view of the measurement apparatus 5 being used in a measurement. The measurement apparatus 5 has a chair-like body having armrests 55L and 55R on both sides of the backrest 51. The armrests 55L and 55R have measuring electrodes 15L and 15R, respectively, which are arranged to contact both palms of the subject B. At the front-side corners of the seat 52, measuring electrodes 14L and 14R are provided, which contact the backs of the knees when the subject B sits down on the seat 52. The measuring electrodes 14L and 14R can be vertically moved by using the lever 59. A footrest 54 having foot-positioning parts 56L and 56R is provided at the place where the feet should be put. In the foot-positioning parts 56L and 56R, current-carrying electrodes 13L and 13R are provided at the fingertip side, and measuring electrodes 36L and 36R are provided at the heel side. A holding plate 57, which can slide in the vertical direction, is provided on the apron 53. The holding plate 57 has measuring electrodes 35L and 35R projecting from the front side, which are arranged to contact the backs of the heels.

As shown in FIG. 35, the foot-positioning part 56L (and 56R) is urged upward by springs 58L fixed to the footrest 54. When, as shown in FIG. 36, the subject sits down on the seat 52 with the feet placed on the foot-positioning parts 56L and 56R, the foot-positioning parts 56L and 56R sink according to the height of the knees from the soles of the feet. Due to the action of the springs 58L, the current-carrying electrodes 13L and 13R and the measuring electrodes 36L and 36R assuredly contact the soles of the feet. Then, by an operation of the lever 59, the measuring electrodes 14L and 14R contact the backs of the knees.

With both feet placed on the foot-positioning parts 56L and 56R, the subject B deeply sits down on the seat 52 and straightens the spine, leaning against the backrest 51. The arms are placed on the armrests 55L and 55R with both palms put on the measuring electrodes 15L and 15R. It is recommended for the subject to open the armpits so that the upper arms do not contact the trunk. This positioning of the subject B provides two current-supplying points Pi1 and Pi2 and four voltage-measuring points Pv1, Pv2, Pv9 and Pv10 as shown in FIG. 10.

The above positioning of the subject B provides the same voltage-measuring points as the measurement apparatus of the third embodiment, so that the measurement can be similarly performed. However, the bending angle of the knee, which is now about 90 degrees, is different from that in the case of the measurement apparatus of the third embodiment. Accordingly, it is preferable to appropriately correct the measurement data, taking into account the influence of the bending state of muscles.

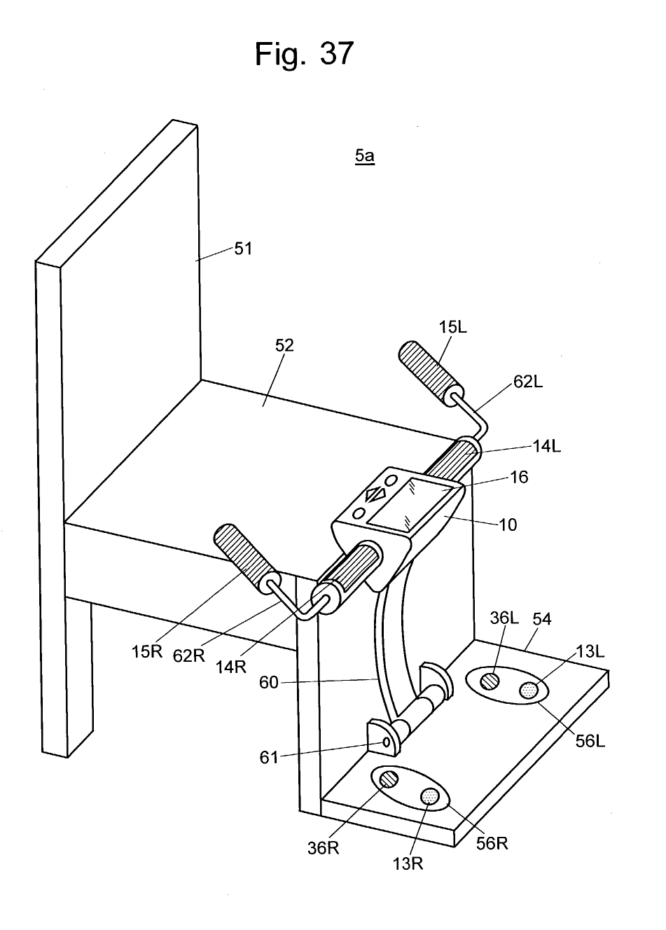

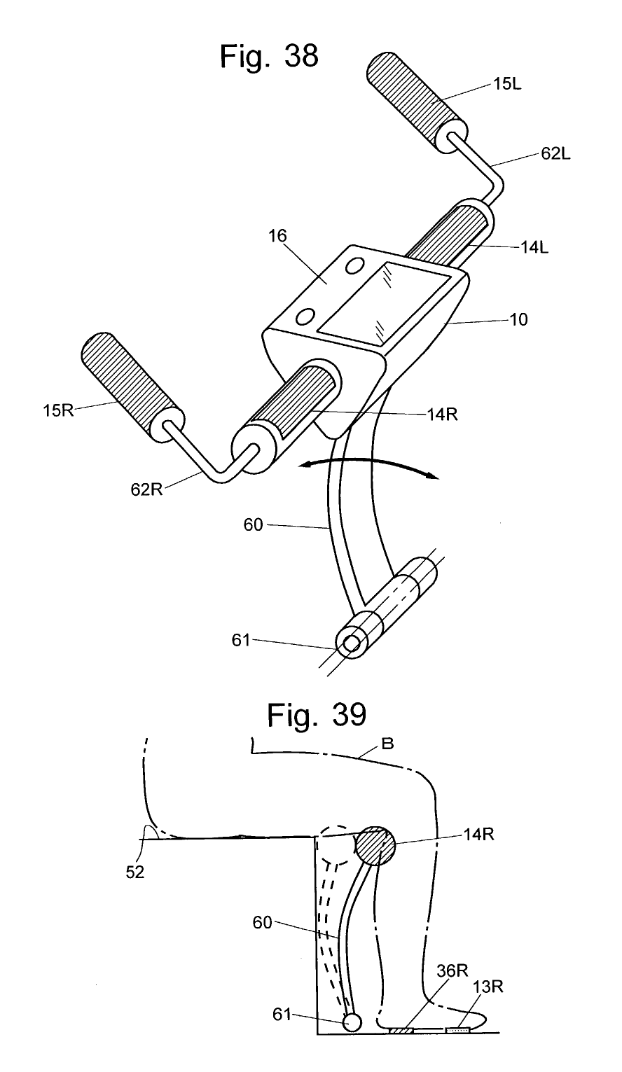

FIG. 37 is a perspective view of a body composition measurement apparatus 5a as a variation of the fifth embodiment, FIG. 38 is an enlarged view of a part of FIG. 38, and FIG. 39 is a partial side view of the measurement apparatus 5a being used in a measurement. In the measurement apparatus 5a, the body 10 having the operation/display panel 16 is fixed to the upper end of a rotary stand 60, which can rotate around a horizontal shaft 61. The body 10 has a pair of L-shaped bars 62L and 62R constructed similar to the bars 17L and 17R in FIG. 11. The subject B sits down on the seat 52 with the feet placed on the foot-positioning parts 56L and 56R of the footrest 54, and with the rotary stand 60 set behind the knees. Next, the subject grips the columnar-shaped measuring electrodes 15L and 15R with both hands, and pushes the rotary stand 60 forward so that the measuring electrodes 14L and 14R contact the backs of the knees. This positioning not only makes the measuring electrodes 14L and 14R contact the backs of the knees but also ensures the contact between the measuring electrodes 15L, 16R and the palms, because the subject B needs to firmly grip the measuring electrodes 15L and 16R to perform the above operation.

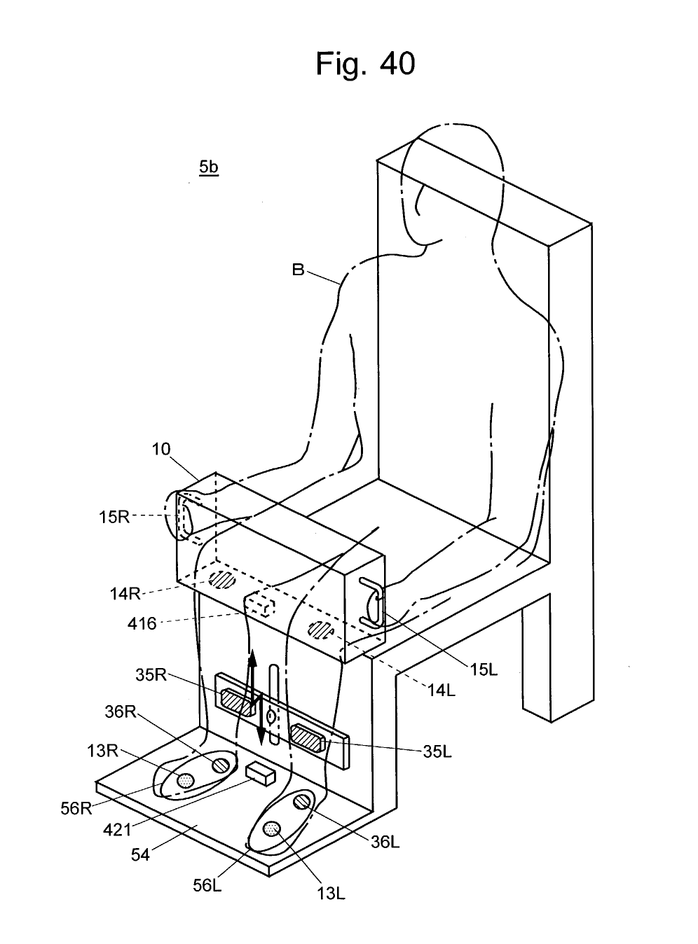

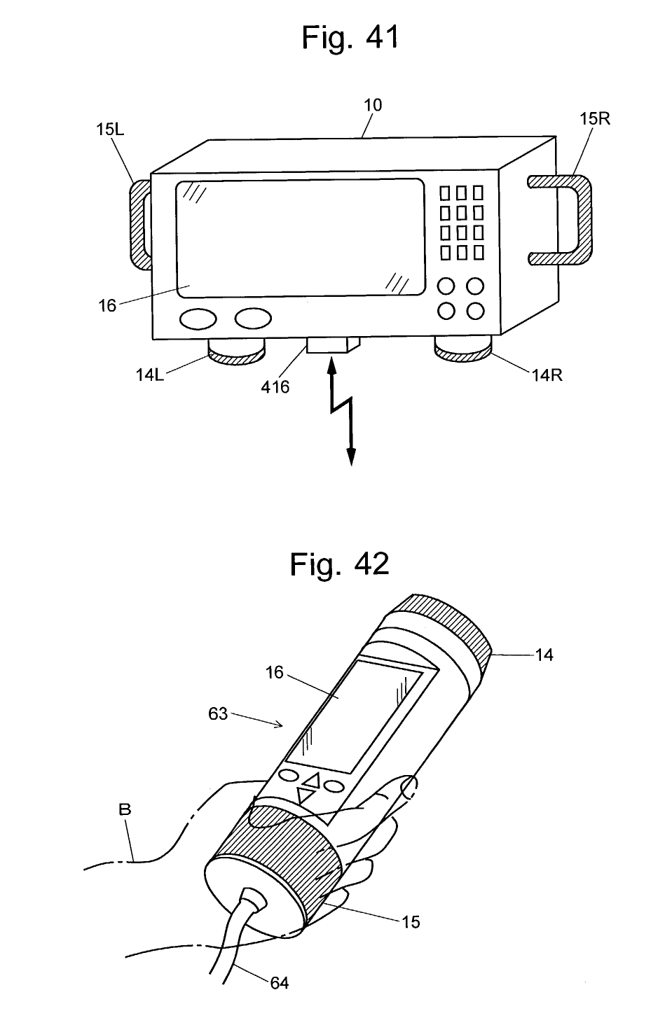

FIG. 40 is a perspective view of a body composition measurement apparatus 5b as another variation of the fifth embodiment, and FIG. 41 is a perspective view of a part of the measurement apparatus 5b. This measurement apparatus 5b is a combination of the body 10 to be placed on the knees, as shown in FIGS. 22 and 23, and a chair having measuring electrodes and current-carrying electrodes to be attached to the heels or soles of the feet. The body 10 and the chair are separated; a wireless communication system, such as infrared communication as described above, is used for the communication between the body 10 and the chair.

[Sixth Embodiment]

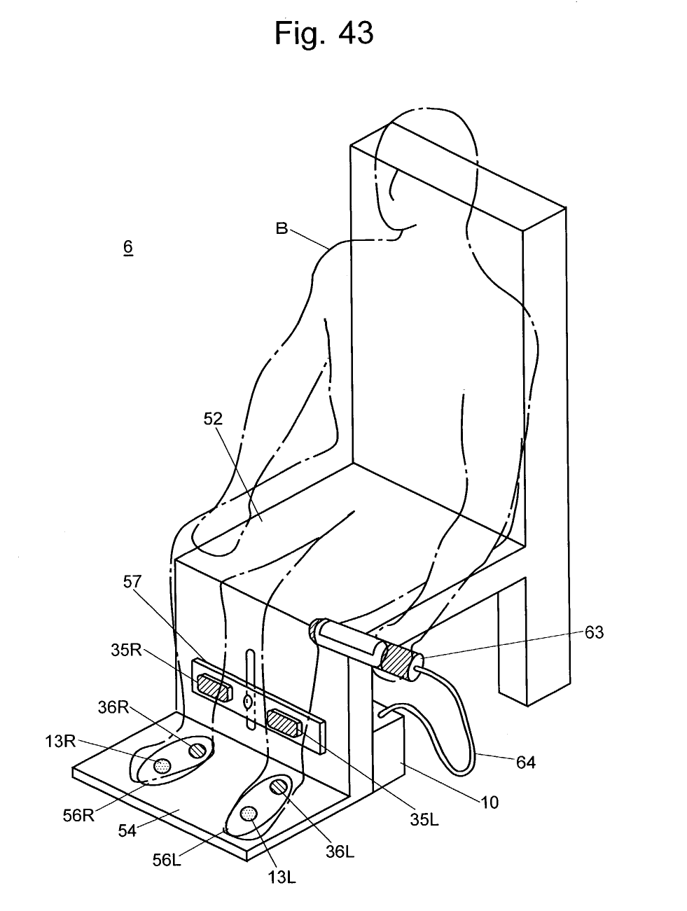

FIG. 43 is a perspective view of a body composition measurement apparatus 6 according to the sixth embodiment of the present invention, being used in a measurement, and FIG. 42 is an enlarged view of a part of the measurement apparatus 6. In this measurement apparatus 6, the constructions of the measuring electrodes 35L, 35R, 36L and 36R arranged to contact the ankles and the soles of the feet, and the current-carrying electrodes 13L and 13R arranged to contact the soles of the feet, are the same as the corresponding elements of the fifth embodiment. On the other hand, the constructions of the measuring electrodes 14L, 14R, 15L and 15R arranged to contact the knees and the palms are different from the fifth embodiment. As shown in FIG. 43, the measurement apparatus 6 has a columnar measurement unit 63 connected by a cable 64 to the body 10 containing main electrical circuits. As shown in FIG. 42, the measurement unit 63 has a measurement electrode 15 on the circumferential side of the part to which the cable is connected, and a measurement electrode 14 at the end of the opposite side. The measurement electrode 15 is arranged to contact a palm, and the measurement electrode 14 is arranged to contact the knee. In addition, the operation/display panel 16 is provided between these electrodes.

The subject B sits down on the seat 52, as shown in FIG. 43, and holds the measurement unit 63 with one hand, as shown in FIG. 42. Then, the palm contacts the measuring electrode 15. In this state, the subject B herself or himself presses the fore end of the measuring unit 63, i.e. the measuring electrode 14, to the knee. In this state, when the operation switch provided on the operation/display panel 16 is pressed, a predetermined measurement is performed. After that, the subject B holds the measurement unit 63 with the other hand, presses it to the other knee, and presses the operation switch again. Thus, in connection with the operation performed by the subject herself or himself, the impedances of predetermined parts of the body can be measured from part to part. Thus, the same measurement result as obtained in the fifth embodiment can be obtained in the end.

Similar to the fourth embodiment, the measurement apparatus 6 according to the sixth embodiment may employ a wireless communication system using light, radio waves, ultrasonic waves, etc., for the communication between the body 10 and the measurement unit 63.

[Seventh Embodiment]

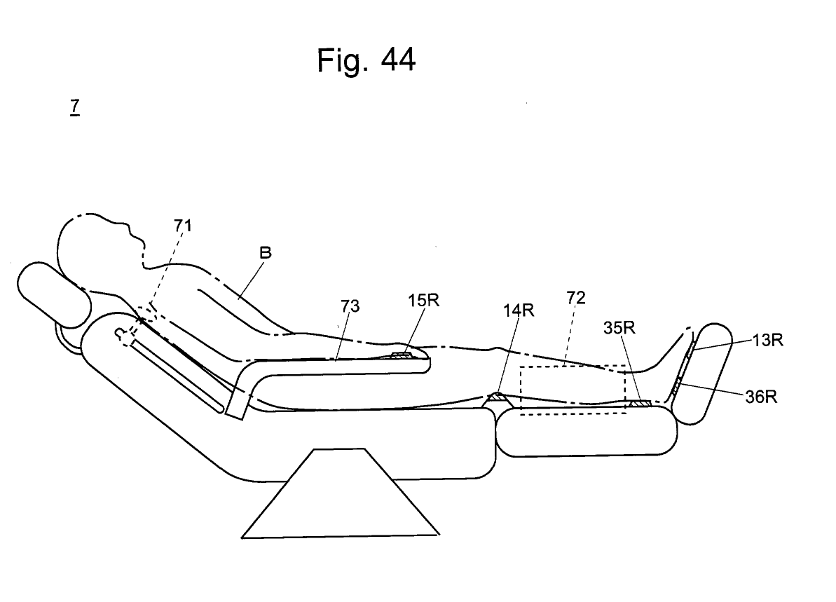

FIG. 44 is a side view of a body composition measurement apparatus 7 according to the seventh embodiment of the present invention. This measurement apparatus 7 is an example of the aforementioned body composition measurement apparatus having a mechanism for giving desirable stimulus to the body of the subject. The measurement apparatus 7 is constructed as a reclining chair provided with massaging function. The reclining chair has a pair of armrests 73L and 73R to support the arms of the subject B, through the left armrest 73L is not shown in FIG. 44. The measuring electrodes 15L and 15R to contact the palms are provided on the upper sides of the ends of the armrests 73L and 73R, respectively, though the left measuring electrode 15L on the left side is not shown in FIG. 44. Furthermore, the measurement apparatus 6 has measuring electrodes 14L, 14R, 35L, 35R, 36L and 36R arranged to contact the backs of the knees, the backs of the ankles and the bottoms of the heels, respectively, and current-carrying electrodes 13L and 13R arranged to contact the soles of the feet at the fingertip side. Again, it should be noted that the left-side electrodes 13L, 14L, 35L and 36L are not shown in FIG. 44. The reclining chair has an upper massager 71 for massaging the body from the back to the shoulders, and a lower massager 72 for massaging for air-massaging the calves.

The combination of the body composition measurement apparatus and the massager makes it possible to measure the muscle mass, right-and-left balance or other information about each part of the body before and after massaging, and to check the improvement in blood circulation inside the muscle or the effect on elimination of swelling.

It should be noted that the embodiments described above are mere examples of the present invention, which can be changed or modified in various ways within the spirit and scope of the present invention.

What is claimed is:

1. A body composition measurement apparatus, comprising:
a) a positioning supporter for supporting a joint located at an end of a predetermined target part of a body of a subject at a preset angle;
b) an electrode holder for fixedly or movably holding plural electrodes so that the electrodes contact predetermined parts of the body of the subject;
c) an impedance-measuring device for measuring an impedance of the target part with the electrodes; and
d) an estimator for estimating various kinds of information about body composition and/or health condition of the target part or entire body of the subject, based on the measured impedance.

2. A body composition measurement apparatus, comprising:
a) a positioning supporter for supporting a joint located at an end of a predetermined target part of a body of a subject at a preset angle;
b) an electrode holder for fixedly or movably holding plural electrodes so that the electrodes contact predetermined parts of the body of the subject;
c) an impedance-measuring device for measuring an impedance of the target part with the electrodes; and
d) an estimator for estimating muscle mass of the target part or entire body of the subject, based on the measured impedance.

3. A body composition measurement apparatus, comprising:
a) a positioning supporter for supporting a joint located at an end of a predetermined target part of a body of a subject at a preset angle;
b) an electrode holder for fixedly or movably holding plural electrodes so that the electrodes contact predetermined parts of the body of the subject;
c) an impedance-measuring device for measuring an impedance of the target part with the electrodes; and
d) an estimator for estimating bone density of the target part or entire body of the subject, based on the measured impedance.

4. The body composition measurement apparatus according to claim 1, wherein the positioning supporter supports the joint at a predetermined angle within a range from about 140 degrees to 180 degrees.

5. The body composition measurement apparatus according to claim 1, wherein the positioning supporter support the joint at an angle of about 90 degrees.

6. The body composition measurement apparatus according to claim 1, wherein the impedance-measuring device is constructed based on an approximate model where the impedance of a part of the body is represented by impedance elements connected in parallel, including impedance elements corresponding to fatty tissue, muscular tissue and osseous tissue, respectively, and where the entire body of a human being is separated into body parts, in which the component ratios of the tissues are fixed and the whole component tissues and each tissue have fixed electrical characteristics, and the impedance-measuring device further includes:
c1) plural current-carrying electrodes and plural measuring electrodes, to be attached to the body of the subject to measure the impedance of the target part composed of one body part or two or more body parts connected in series;
c2) a current-supplying device for supplying an alternating current of a preset frequency via the current-carrying electrodes through at least the target part;
c3) a voltage-measuring device for measuring a voltage induced by the alternating current over the target part; and
c4) a calculator for calculating the impedance corresponding to the target part from the voltage measured and a current value of the alternating current.

7. The body composition measurement apparatus according to claim 6, wherein the estimator d) is constructed to estimate the muscle mass, bone density or various kinds of information relating to the body composition or health condition of the target part or entire body of the subject, using:
a first estimation formula created based on a result of a measurement of the impedance for the entire body and/or each body part of each of plural pretest subjects, and based on body composition information of the entire body and/or each body part of each of the pretest subjects obtained by observing an inside of the entire body and/or each body part of each of the pretest subjects; or
a second estimation formula created by adding the body identification information of the pretest subjects to the first estimation formula.

8. The body composition measurement apparatus according to claim 7, wherein the estimator d) obtains body composition information of the entire body and/or a part of the body of the subject by a measurement using an apparatus capable of acquiring cross-sectional images of the human body.

9. The body composition measurement apparatus according to claim 1, further comprising a body identification information acquirer for acquiring body identification information of the subject, and the estimator estimates the muscle mass, bone density or various kinds of information relating to the body composition or health condition of the target part or entire body of the subject, based on the measured impedance and the body identification information.

10. The body composition measurement apparatus according to claim 9, wherein the body identification information acquirer includes a partial size estimator for estimating a predetermined size or sizes of the target part based on a height of the subject given as one item of the body identification information, or further taking account of weight, age, sexuality or other information, and adding the estimated size or sizes to the body identification information.

11. The body composition measurement apparatus according to claim 10, wherein the body identification information acquirer includes a size measurer for measuring an actual size of the target part of the subject.

12. The body composition measurement apparatus according to claim 1, wherein the joint includes at least a joint of a knee.

13. The body composition measurement apparatus according to claim 12, wherein the positioning supporter supports lower limbs at least at backs of the knees when the subject is in a sitting position with the lower limbs stretched forward.

14. The body composition measurement apparatus according to claim 13, wherein the positioning supporter includes a trapezoidal or triangular flat body having slopes for resting the lower limbs.

15. The body composition measurement apparatus according to claim 13, wherein the positioning supporter includes an approximately horizontal bar located at a predetermined level for supporting the backs of the knees.

16. The body composition measurement apparatus according to claim 13, wherein the positioning supporter is constructed like a chair having a seat for the subject to sit down, where a level of the seat is determined so that the knees of the subject are bent to an almost right angle while soles of feet are placed on a floor or other approximately horizontal plane equivalent to the floor.

17. The body composition measurement apparatus according to claim 1, wherein the electrodes include a pair of measuring electrodes that contact a proximity to knees of the subject, at least one measuring electrode that contacts a trunk or upper limb of the subject, and a pair of current-carrying electrodes that contact the body at two points located farther than the knees from the trunk of the subject.

18. The body composition measurement apparatus according to claim 17, wherein said at least one measuring electrode that contacts the trunk or upper limb contacts a palm of the subject.

19. The body composition measurement apparatus according to claim 13, wherein the estimator estimates at least muscle mass of a thigh of the subject or other body composition information correlated with the muscle mass of the thigh.

20. The body composition measurement apparatus according to claim 13, wherein the estimator estimates at least a balance of muscle mass of both thighs of the subject or other body composition information correlated with the above balance.

21. The body composition measurement apparatus according to claim 19, wherein the estimator uses a length of the thigh estimated based on the body identification information.

22. The body composition measurement apparatus according to claim 17, wherein the current-carrying electrodes contact both shanks of the subject.

23. The body composition measurement apparatus according to claim 17, wherein the measuring electrodes that contact a proximity to knees are arranged to contact backs of the knees.

24. The body composition measurement apparatus according to claim 23, wherein the measuring electrodes that contact a proximity to knees are located at a top the positioning supporter having a trapezoidal or triangular flat body with slopes for resting the lower limbs, and the current-carrying electrodes that contact the shanks are located on the slope.

25. The body composition measurement apparatus according to claim 23, wherein the measuring electrodes that contact a proximity to knees are located on an upper side of the horizontal bar for supporting the knees, and the current-carrying electrodes are arranged to contact the backs of the calves at a level lower than the bar.

26. The body composition measurement apparatus according to claim 17, wherein the measuring electrodes that contact a proximity to knees are arranged to contact surfaces of the knees.

27. The body composition measurement apparatus according to claim 17, wherein the measuring electrodes that contact a proximity to knees are arranged to contact insides of the knees when the apparatus is held between the knees.

28. The body composition measurement apparatus according to claim 18, wherein the electrode that contacts a palm is a grip-like electrode to be gripped by the subject, which serves as the electrode holder with a handle for pulling the grip-like electrode out from a body of the apparatus.

29. The body composition measurement apparatus according to claim 18, wherein the measuring electrode that contacts a palm is a grip-like electrode, which serves as the electrode holder when connected to the body by a cable.

30. The body composition measurement apparatus according to claim 18, wherein the measuring electrode that contacts a palm is provided on each of both sides of the body of the apparatus so that the measuring electrodes contact both palms when the subject touches both sides of the body of the apparatus with both hands.

31. The body composition measurement apparatus according to claim 1, wherein the electrode holder includes a position adjuster for adjusting contact positions of the measuring electrodes.

32. The body composition measurement apparatus according to claim 31, wherein the position adjuster includes a size-measuring device for measuring a size or sizes of the target part according to the contact positions adjusted.

33. The body composition measurement apparatus according to claim 1, wherein the apparatus includes a square-shaped body housing containing an electrical circuit, and the measuring electrodes are arranged at preset intervals on both sides, bottom side or other side adjacent to the aforementioned sides of the body housing so that the measurement can be performed with the knees set apart from each other by a preset distance.

34. The body composition measurement apparatus according to claim 33, wherein the body housing is provided with a display on its front side or upper side.

35. The body composition measurement apparatus according to claim 1, wherein the electrode holder is constructed to allow the subject to grip and move the electrode holder to any position on the body, and the impedance-measuring device measures the impedance elements corresponding to different contact parts of the measuring electrode held by the electrode holder.

36. The body composition measurement apparatus according to claim 1, wherein the impedance-measuring device includes a first unit for principally measuring upper limbs of the subject and a second unit for principally measuring lower limbs of the subject, and a cable connects the first and second units for transferring signals between the two units.

37. The body composition measurement apparatus according to claim 1, wherein the impedance-measuring device includes a first unit for principally measuring upper limbs of the subject and a second unit for principally measuring lower limbs of the subject, and a wireless communication system is used to transfer signals between the first and second units.

38. The body composition measurement apparatus according to claim 1, wherein the positioning supporter includes a stimulating device for giving a stimulus to at least a part of the body of the subject.

39. The body composition measurement apparatus according to claim 38, wherein the stimulating apparatus is a massager for massaging at least a part of the body of the subject.

40. The body composition measurement apparatus according to claim 39, wherein the positioning supporter is constructed as a chair having the massager.
